(19) United States
(12) Patent Application Publication
(10) Pub. No.: US 2007/0270455 A1
(43) Pub. Date: Nov. 22, 2007

(54) INHIBITION OF NF-kB

(76) Inventors: Andrei V. Gudkov, Gates Mills, OH (US); Katerina V. Gurova, Highland Heights, OH (US)

Correspondence Address: POLSINELLI SHALTON FLANIGAN SUELTHAUS PC, 700 W. 47TH STREET, SUITE 1000, KANSAS CITY, MO, 64112-1802, US

(21) Appl. No.: 11/624,828

(22) Filed: Jan. 19, 2007

(30) Foreign Application Priority Data
Jul. 20, 2005 (US) PCT/US05/25884

(51) Int. Cl. A61K 31/473; A61P 35/04; G01N 33/566

(52) U.S. Cl. 514/297; 435/7.21

(57) ABSTRACT

Aminoacridines are inhibitors of NF-κB. Inhibiting NF-κB leads to reactivation of p53 in cancer cells with functionally blocked p53.

### CROSS-REFERENCE TO RELATED APPLICATIONS

The present application is a continuation of International Application No. PCT/US2005/025884, filed Jul. 20, 2005, which claims the benefit of U.S. Provisional Application No. 60/589,637, filed Jul. 20, 2004, the contents each of which are incorporated herein by reference.

### BACKGROUND OF THE INVENTION

1. Field of the Invention

The present invention is generally related to the modulation of cell growth or apoptosis. More specifically, the present invention is related to compositions for modulating cell growth or apoptosis, methods of use thereof, and methods of identification thereof.

2. Description of Related Art

The frequency of cancer in humans has increased in the developed world as the population has aged. For some types of cancers and stages of disease at diagnosis, morbidity and mortality rates have not improved significantly in recent years in spite of extensive research. Induction of programmed cell death or apoptosis is one of the most attractive cancer treatment strategies.

p53 controls genetic stability and reduces the risk of cancer through induction of growth arrest or apoptosis in response to DNA damage or deregulation of proto-oncogenes. The efficacy of p53 as a tumor-preventing factor is reflected by the frequency of p53 loss in at least 50% of human tumors due to inactivating mutations. Several mechanisms of functional inactivation of wild type p53 have been described in human tumors, usually involving excessive degradation of p53 via proteasomes and mediated by Mdm2. Mdm2 is considered an attractive target for suppression by small molecules or other approaches in order to selectively kill tumor cells by restoring p53 function.

Renal cell carcinomas (RCC) maintain wild type but functionally inactive p53. The mechanism of p53 repression in RCC is dominant, which indicates the existence of a so far unknown molecular target for restoration of p53 function in cancer. There is a significant need to identify agents that are capable of restoring wild type p53 activity in tumor cells.

### SUMMARY OF THE INVENTION

A condition associated with NF-κB activity may be treated by administering to a patient in need thereof a composition comprising an inhibitor of NF-κB. The NF-κB activity may be constitutive, induced or at a basal level. The inhibition of NF-κB may activate p53. The inhibition of NF-κB may activate functionally silent p53. The condition treated may be cancer, inflammation, autoimmune disease, graft versus host disease, a condition associated with HIV infection, or pre-cancerous cells which have acquired dependence on constitutively active NF-κB. Forms of cancer, which may be treated, include, but are not limited to, renal cell carcinoma, sarcoma, prostate cancer, breast cancer, pancreatic cancer, myeloma, myeloid and lymphoblastic leukemia, neuroblastoma, glioblastoma or a cancer caused by HTLV infection.

The inhibitor of NF-κB may be an aminoacridine of the formula: 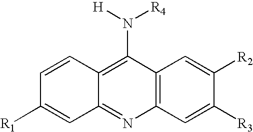
wherein,
R₁ is H or halogen;
R₂ is H or optionally substituted alkoxy;
R₃ is H or optionally substituted alkoxy; and
R₄ is H or optionally substituted aliphatic, aryl, or heterocycle.
   The aminoacridine may be 9-aminoacridine or quinacrine. The composition may further comprise an activator of a death receptor of a TNF family polypeptide. The activator may be a TNF polypeptide, such as NGF, CD40L, CD137L/4-1BBL, TNF-α, CD134L/OX40L, CD27L/CD70, FasL/CD95, CD30L, TNF-β/LT-α, LT-β, or TRAIL.

An agent that modulates functionally silent p53 may be identified by adding a candidate agent to a cell comprising a p53-responsive reporter and measuring the level of signal of the p53-responsive reporter. The agent may be identified by a difference in the signal compared to a control. The agent may increase or decrease the activity of p53. The cell may comprise a functionally silent p53.

An agent that modulates NF-κB may be identified by adding a candidate agent to a cell comprising a p53-responsive reporter and measuring the level of signal of the p53-responsive reporter. The agent may be identified by a difference in the signal compared to a control. The agent may increase or decrease the activity of NF-κB. The cell may comprise a functionally silent p53. The cell may also comprise an NF-κB transactivation complex.

### BRIEF DESCRIPTION OF THE DRAWINGS

FIG. 1 indicates that the restoration of p53-mediated transactivation in RCC cells is accompanied by death of RCC cells. FIG. 1A: p53-responsive reporter activity in RCC45ConALacZ cells transduced with different concentration of p53 or GFP expressing lentiviral vectors. β-galactosidase activity (ONPG staining) was measured 48 hours after lentiviral transduction and normalized by protein concentration. FIG. 1B: Cell survival was measured at 96 hours after lentiviral transduction by methylene blue staining and presented as a percentage of intensity of methylene blue staining of cell transduced with p53-virus to the same cells transduced with the same concentration of GFP-virus.

FIG. 2 indicates the p53 restoration activity of agents of the formula of compound 1. FIG. 2A: Choice of readout cells and setting of selection criterion. MCF7, ACHN, RCC26b and RCC45 cells all containing ConALacZ reporter were plated into 96 well plates and incubated in the medium containing different concentrations of doxorubicin for 24 hours. Then β-galactosidase activity was measured by ONPG staining and normalized by protein concentration. FIG. 2B indicates that 9AA causes the strongest activation of p53-dependent reporter in RCC45 cells. Agents of the formula of compound 1 were tested in dose dependent assay on p53 transactivation in RCC45ConALacZ cells. Bars represent relative activity of each compound calculated as a fold of p53 activation, induced by a compound, over the effect of 2 μM of doxorubicin (results of three experiments).

FIG. 3 indicates that 9AA induces p53 transcriptional activity in different tumor cells. FIG. 3A indicates that 9AA induces p53-responsive reporters in a dose-dependent manner. RCC45ConALacZ and MCF7ConALacZ cells were incubated in media containing indicated concentrations of doxorubicin (dox) or 9AA (all concentrations are presented in μM) for 24 hours and then β-galactosidase activity was measured by ONPG staining, normalized by protein content and presented as a fold of reporter induction compared with untreated cells. FIG. 3B indicates that 9AA induces expression of endogenous p53-dependent targets. Western blot analysis of total cell lysates of RCC45 and MCF7 cells treated with 10 μM of 9AA or 2 μM of doxorubicin for indicated periods of time (hours) and probed with anti-p53, anti-Hdm2 or anti-p21 antibodies. FIG. 3C indicates that 9AA activates p53 stronger than doxorubicin in the majority of tumor cells tested. Indicated cells with integrated p53 responsive reporter were treated with different concentrations of 9AA (1-10 μM) and doxorubicin (dox, 0.2-2 μM) for 24 hours and then β-galactosidase activity was measured by ONPG staining and normalized by protein content. Data presented as a fold of reporter induction over untreated controls by the most effective dose of 9AA over effect of dox. FIG. 3D indicates that 9AA induces β-galactosidase activity in a p53-dependent manner. HT1080ConALuC transduced with anti-p53 or anti-GFP siRNA expressing constructs were treated with 5 μM of 9AA for 24 hours. Bars represent folds of p53 responsive reporter induction over untreated controls. Box represents western blot analysis of p53 expression in total protein lysates of both cells variants in basal and doxorubicin treated conditions (to evaluate basal and DNA-damage induced level of p53). FIG. 3E shows a dose-response curve of p53-responsive reporter activity in HT1080ConALuc cells treated with 9AA for 24 hours (no normalization for protein concentration was done). Fold induction presented as fold of reporter activation over untreated control. FIG. 3F shows the time dependence of the p53-inducing effect of 9AA. HT1080ConALuC cells were treated for 1 hour with 20 μM of 9AA and then β-galactosidase activity was measured at the indicated time points. Fold induction presented as fold of reporter activation over untreated control.

FIG. 4 indicates that 9AA-associated cytotoxicity is p53-dependent. FIG. 4A shows the survival of HT1080-sip53 and HT1080-siGFP cells treated with indicated concentrations of 9AA (see Material and Methods) with results presented as a percentage of cells compared with an untreated control. FIG. 4B shows other pairs of cells with different levels of p53 tested in the manner described for FIG. 4A. The upper panel shows a western blot analysis of p53 protein level in the generated pairs of cells. The lower panel shows the relative number of cells after treatment with 9aa treatment (2 μM) compared to an untreated control (100%). FIG. 4C shows a cell cycle analysis of HT1080 sip53 or HT1080 siGFP cells treated with 3 or 20 μM of 9AA during the indicated periods of time or 2 μM of dox during 24 hours. FIG. 4D shows the p53-dependence of the cytotoxicity of different drugs. The same experiment as described in FIG. 4A was done using 30d9 (primary hit, analogue of 9aa, 1-10 μM) doxorubicin (dox, 0.1-1 μM), campothecin (camp, 0.16-1.6 μM), vinblastin (vinbl, 0.1-1 μM) and taxol (tax, 0.06-0.6 μM). Bars are plotted for the dose of drugs, demonstrating the highest difference in sensitivity between p53 “plus” and “minus” cells. FIG. 4E indicates that 9AA is more toxic for RCC cells than for normal kidney epithelial cells (NKE). The same experiment as described in FIG. 4A was performed with NKE, RCC45, RCC54 and ACHN cells. FIG. 4F indicates that 9AA is more toxic for RCC cells at low concentrations. Several cell types (NKE,—normal kidney epithelial cells, RCC45, ACHN—RCC cell lines, HCT116—colon carcinoma, p53 wild type, SK-N-SH—neuroblastoma, p53 wild type, LNCaP prostate adenocarcinoma, p53 wild type, DU145, PC3—prostate adenocarcinoma, p53 deficient, Mel7, Mel29 melanomas, 041—fibroblasts from patient with Li-Fraumeni syndrome, p53-null, WI38—normal human diploid fibroblasts) were treated with 2 μM of 9AA as described in FIG. 4A and cell survival was compared with the corresponding untreated cells.

FIG. 5 shows that agents of the formula of compound 1 are toxic for tumor cells with active p53. FIG. 5A shows the p53 inducing effects of agents in vivo. HT1080ConALuC cells were inoculated into two flanks of nude mice. When tumors reach 5 mm in diameter, mice were injected intraperitoneally with indicated concentrations of the drugs (mg/kg, 3 mice per group). After 24 hours mice were sacrificed, tumors were isolated, lysed in Reporter Lysis Reagent (Promega) and luciferase activity was measured in 10 mg of tumor proteins. Bars represent fold of induction of luciferase activity in tumors, treated with drugs over luciferase activity in tumors treated with vehicle. FIG. 5B: HT1080sip53 or HT1080siGFP cells were inoculated in the left and right flank of nude mice respectively. When tumors reached 5 mm in diameter, mice were injected intraperitoneally with vehicle (50% DMSO in PBS), quinacrine (QC, 50 mg/kg) and 5-fluorouracil (5FU, 35 mg/kg) every 24 hours (5 mice per group). Results are presented as medians of relative tumor volume for each tumor comparing with the volume of tumor in the first day of treatment.

FIG. 6 shows the testing of the potential mechanism of 9AA activity. FIG. 6A shows the measurement of DNA—topoisomerase II complex formation in cells treated with 9-AA. FIG. 6B shows the p53 phosphorylation status in RCC45 cells treated with 9AA (5 μM) or dox (1 μM) for 16 hours. Western blot analysis of total protein lysates was performed using antibodies against p53 (DO1) and against specific sites of phosphorylation in p53. FIG. 6C shows the effect of 9AA on proteasome activity. HCT116 cells were treated for 30 minutes with 1 μM PS-341. PS-341 was washed off after 30 minutes and cells were re-incubated in drug free medium prior to analysis at 3 hours or 16 hours post-treatment. In the case of 9-AA, cells were treated continuously with 2 μM 9-aminoacridine for 3 hours or 16 hours prior to analysis. Proteasome activity was determined by measuring the absorbance of free AMC generated by cleavage of an AMC-fluorogenic peptide. The proteasome activity of untreated cells was set at 100%. FIG. 6D shows the status of IκB-α phosphorylation after treatment with 9-AA. PC-3 cells were treated with 10 μM 9-aminoacridine for 1, 2, 4, and 8 hours or with 10 μM MG-132 for 8 hours. Cell lysates were isolated at the indicated hours after treatment and used for western blotting. Blots were probed with antibodies specific for both phosphorylated and unphosphorylated forms of IκB-α. β-actin specific antibodies were used as a loading control. FIG. 6E shows that 9AA treatment leads to a decrease in IκB-α protein levels. Western blot analysis is shown of MCF7 cells incubated in 1 μM of doxorubicin (dox) and 10 μM of 9AA during 8 hours with anti IκB antibodies.

FIG. 7 shows the effects of 9AA on the NF-κB pathway. FIG. 7A shows that 9AA inhibits NF-κB-dependent transcription. H1299-NF-kBLuc cells were treated with different concentrations of 9AA and quinacrine (QC) two hours before (TNF after 9AA or QC) or simultaneously (TNF and 9AA or QC) with TNFa (10 ng/ml). 6 hours after addition of TNFa luciferase activity was measured in cell lysates. FIG. 7B shows that 9AA inhibits reconstitution of IκB levels stimulated by TNF. HT1080 cells were treated with TNF (10 ng/ml) in the presence or absence of 9AA (10 μM). At the indicated time points total cell lysates were prepared and analyzed by western blotting with antibodies against IκB. FIG. 7C: H1299-NF-kBLuc cells were treated with indicated concentrations of 9AA and TNF (10 ng/ml). After 6 hours, cytoplasmic and nuclear extracts were isolated and used for luciferase or gel-shift assay, respectively. FIG. 7D shows that 9AA causes accumulation of p65/p50 and p50/p50 NF-κB complexes. Gel-shift assays were performed with nuclear extracts of H1299 cells, treated with 9AA (10 μM) and TNF (10 ng/ml) for 30 minutes. FIG. 7E shows that 9AA retards exit of p65 complexes from the nuclei. Immunofluorescent staining of HT1080 cells treated with 9AA (10 mM) and TNF (10 ng/ml) during indicating periods of time with antibodies against p65. FIG. 7F shows that 9AA decreases phosphorylation of p65 in response to TNF. Upper panel: western blot analysis of total cell lysates of HT1080 cells, treated with TNF (10 ng/ml) in the presence or absence of 9AA (10 μM) during indicated periods of time. The same membranes were probed with antibodies against total p65 and against phospho-p65Ser536. Lower panel: quantitation of experiment, presented in the upper panel using BioRad QuantityOne software. Results are presented as a fold of changing in the intensity of bands, comparing with untreated control. FIG. 7G shows that 9AA causes increase in p50 protein level. Western blot analysis of nuclear and cytoplasmic fractions of HT1080 cells; treated as described in FIG. 7A. FIG. 7H shows that 9AA does not inhibit NF-κB transactivation induced by trichostatin A (TSA). H1299 cells with integrated NF-κB-dependent luciferase reporter were treated with 100 nM of TSA in the presence or absence of 9AA (20 mM) for 4 or 16 hours. TNFa treatment was used as a control of reporter activity.

FIG. 8 shows that 9AA activates p53-dependent transcription through inhibition of NF-κB. FIG. 8A shows that IκB SuperSupressor (ss) activates p53 in RCC cells. ACHN cells were cotransfected with p21-ConALuc and indicated plasmids, containing IkB SuperSuppressor, p53 and Arf cDNA or anti-Hdm2 siRNA. Forty-eight hours later, luciferase activity was measured in cell lysates. Normalization was done by cotransfection of the pCMV-LacZ plasmid. FIG. 8B shows that IκB SuperSupressor inhibits NF-κB transcriptional activity. ACHN cells were cotransfected with the NF-κB-responsive reporter pNF-κBLuc and IκB Super Suppressor (SS). Forty-eight hours later luciferase activity was measured in cell lysates. FIG. 8C shows that 9AA cannot activate p53 in cells with inhibited NF-κB. ACHN cells were cotransfected with either pConALuc or pNF-κBLuc and IκB Super Supressor (SS) or empty vector. Twenty-four hours post-transfection all cells were split and treated with 9AA (10 μM). NF-κB reporter activity was measured 6 hours post-treatment and p53 responsive reporter activity was measured 24 hours after treatment. Normalization was done by cotransfection of pCMV-LacZ plasmid (for cells, transfected by different set of plasmids) and by protein concentrations (for 9AA treated and untreated cells).

FIG. 9 shows the synergistic effect of 9-aminoacridine with death ligands.

FIG. 10 shows that 9AA and QC are anti-RCC agents. FIG. 10A shows a comparison of IC50% doses of 9AA, QC and several anti-cancer agents of different RCC and non-RCC cells. IC50% for each cell line and each drug was determined. Each point represents IC50% of particular cell line, which are grouped as follows: (i) black circles—RCC cell lines (ACHN, RCC9, RCC13, RCC29, RCC45, RCC54), (ii) red triangles—non-RCC cell lines (MCF7, HT1080, H1299, U20S, LNCaP, HCT116), (iii) green squares—normal kidney cells (NKE). FIG. 10B shows that quinacrine activates p53-responsive reporter in ex vivo-cultured RCC tumors. X-gal staining of tumor and normal kidney pieces transduced with p53-responsive reporter lentivirus ex vivo and treated with quinacrine or doxorubicin. FIG. 10C shows that quinacrine sensitizes RCC45 and RCC54 but not normal kidney epithelium (NKE) to TRAIL. Cells pleated in 96-well plates were incubated 24 hours in the presence of indicated concentrations of TRAIL and quinacrine; cell numbers were estimated using methylene blue assay. FIG. 10D shows the anti-tumor activity of quinacrine (QC). 10⁷ of ACHN cells were inoculated under the skin of nude mice. At the moment tumors achieved 5 mm in diameter QC administrations were started intraperitoneally, 50 mg/kg. 5FU (35 mg/kg) was used as a control. Tumor size was measured every other day and presented as a fold increase in tumor volume.

### DETAILED DESCRIPTION

Before the present compounds, products and compositions and methods are disclosed and described, it is to be understood that the terminology used herein is for the purpose of describing particular embodiments only and is not intended to be limiting. It must be noted that, as used in the specification and the appended claims, the singular forms “a,” “an” and “the” include plural referents unless the context clearly dictates otherwise.

1. Definitions

The term “branched” as used herein refers to a group containing from 1 to 24 backbone atoms wherein the backbone chain of the group contains one or more subordinate branches from the main chain. Preferred branched groups herein contain from 1 to 12 backbone atoms. Examples of branched groups include, but are not limited to, isobutyl, t-butyl, isopropyl, —CH₂CH₂CH(CH₃)CH₂CH₃, —CH₂CH(CH₂CH₃)CH₂CH₃, —CH₂CH₂C(CH₃)₂CH₃, —CH₂CH₂C(CH₃)₃ and the like.

The term “unbranched” as used herein refers to a group containing from 1 to 24 backbone atoms wherein the backbone chain of the group extends in a direct line. Preferred unbranched groups herein contain from 1 to 12 backbone atoms.

The term “cyclic” or “cyclo” as used herein alone or in combination refers to a group having one or more closed rings, whether unsaturated or saturated, possessing rings of from 3 to 12 backbone atoms, preferably 3 to 7 backbone atoms.

The term “lower” as used herein refers to a group with 1 to 6 backbone atoms.

The term “saturated” as used herein refers to a group where all available valence bonds of the backbone atoms are attached to other atoms. Representative examples of saturated groups include, but are not limited to, butyl, cyclohexyl, piperidine and the like.

The term “unsaturated” as used herein refers to a group where at least one available valence bond of two adjacent backbone atoms is not attached to other atoms. Representative examples of unsaturated groups include, but are not limited to, —CH₂CH₂CH═CH₂, phenyl, pyrrole and the like.

The term “aliphatic” as used herein refers to an unbranched, branched or cyclic hydrocarbon group, which may be substituted or unsubstituted, and which may be saturated or unsaturated, but which is not aromatic. The term aliphatic further includes aliphatic groups, which comprise oxygen, nitrogen, sulfur or phosphorous atoms replacing one or more carbons of the hydrocarbon backbone.

The term “aromatic” as used herein refers to an unsaturated cyclic hydrocarbon group having 4n+2 delocalized π(pi) electrons, which may be substituted or unsubstituted. The term aromatic further includes aromatic groups, which comprise a nitrogen atom replacing one or more carbons of the hydrocarbon backbone. Examples of aromatic groups include, but are not limited to, phenyl, naphthyl, thienyl, furanyl, pyridinyl, (is)oxazoyl and the like.

The term “substituted” as used herein refers to a group having one or more hydrogens or other atoms removed from a carbon or suitable heteroatom and replaced with a further group. Preferred substituted groups herein are substituted with one to five, most preferably one to three substituents. An atom with two substituents is denoted with “di,” whereas an atom with more than two substituents is denoted by “poly.” Representative examples of such substituents include, but are not limited to aliphatic groups, aromatic groups, alkyl, alkenyl, alkynyl, aryl, alkoxy, halo, aryloxy, carbonyl, acryl, cyano, amino, nitro, phosphate-containing groups, sulfur-containing groups, hydroxyl, alkylcarbonyloxy, arylcarbonyloxy, alkoxycarbonyloxy, aryloxycarbonyloxy, alkylcarbonyl, arylcarbonyl, alkoxycarbonyl, aminocarbonyl, alkylaminocarbonyl, dialkylaminocarbonyl, alkylthiocarbonyl, acylamino, amidino, imino, alkylthio, arylthio, thiocarboxylate, alkylsulfinyl, trifluoromethyl, azido, heterocyclyl, alkylaryl, heteroaryl, semicarbazido, thiosemicarbazido, maleimido, oximino, imidate, cycloalkyl, cycloalkylcarbonyl, dialkylamino, arylcycloalkyl, arylcarbonyl, arylalkylcarbonyl, arylcycloalkylcarbonyl, arylphosphinyl, arylalkylphosphinyl, arylcycloalkylphosphinyl, arylphosphonyl, arylalkylphosphonyl, arylcycloalkylphosphonyl, arylsulfonyl, arylalkylsulfonyl, arylcycloalkylsulfonyl, combinations thereof, and substitutions thereto.

The term “unsubstituted” as used herein refers to a group that does not have any further groups attached thereto or substituted therefor.

The term “alkyl” as used herein alone or in combination refers to a branched or unbranched, saturated aliphatic group. Representative examples of alkyl groups include, but are not limited to, methyl, ethyl, n-propyl, isopropyl, n-butyl, isobutyl, sec-butyl, tert-butyl, octyl, decyl, tetradecyl, hexadecyl, eicosyl, tetracosyl and the like.

The term “alkenyl” as used herein alone or in combination refers to a branched or unbranched, unsaturated aliphatic group containing at least one carbon-carbon double bond which may occur at any stable point along the chain. Representative examples of alkenyl groups include, but are not limited to, ethenyl, E- and Z-pentenyl, decenyl and the like.

The term “alkynyl” as used herein alone or in combination refers to a branched or unbranched, unsaturated aliphatic group containing at least one carbon-carbon triple bond which may occur at any stable point along the chain. Representative examples of alkynyl groups include, but are not limited to, ethynyl, propynyl, propargyl, butynyl, hexynyl, decynyl and the like.

The term “aryl” as used herein alone or in combination refers to a substituted or unsubstituted aromatic group, which may be optionally fused to other aromatic or non-aromatic cyclic groups. Representative examples of aryl groups include, but are not limited to, phenyl, benzyl, naphthyl, benzylidine, xylyl, styrene, styryl, phenethyl, phenylene, benzenetriyl and the like.

The term “alkoxy” as used herein alone or in combination refers to an alkyl, alkenyl or alkynyl group bound through a single terminal ether linkage. Examples of alkoxy groups include, but are not limited to, methoxy, ethoxy, n-propoxy, iso-propoxy, n-butoxy, 2-butoxy, tert-butoxy, n-pentoxy, 2-pentoxy, 3-pentoxy, isopentoxy, neopentoxy, n-hexoxy, 2-hexoxy, 3-hexoxy, 3-methylpentoxy, fluoromethoxy, difluoromethoxy, trifluoromethoxy, chloromethoxy, dichloromethoxy, and trichloromethoxy.

The term “aryloxy” as used herein alone or in combination refers to an aryl group bound through a single terminal ether linkage.

The term “halogen,” “halide” or “halo” as used herein alone or in combination refers to fluorine “F”, chlorine “Cl”, bromine “Br”, iodine “I”, and astatine “At”. Representative examples of halo groups include, but are not limited to, chloroacetamido, bromoacetamido, idoacetamido and the like.

The term “hetero” as used herein combination refers to a group that includes one or more atoms of any element other than carbon or hydrogen. Representative examples of hetero groups include, but are not limited to, those groups that contain heteroatoms including, but not limited to, nitrogen, oxygen, sulfur and phosphorus.

The term “heterocycle” as used herein refers to a cyclic group containing a heteroatom. Representative examples of heterocycles include, but are not limited to, pyridine, piperadine, pyrimidine, pyridazine, piperazine, pyrrole, pyrrolidinone, pyrrolidine, morpholine, thiomorpholine, indole, isoindole, imidazole, triazole, tetrazole, furan, benzofuran, dibenzofuran, thiophene, thiazole, benzothiazole, benzoxazole, benzothiophene, quinoline, isoquinoline, azapine, naphthopyran, furanobenzopyranone and the like.

The term “carbonyl” or “carboxy” as used herein alone or in combination refers to a group that contains a carbon-oxygen double bond. Representative examples of groups which contain a carbonyl include, but are not limited to, aldehydes (i.e., formyls), ketones (i.e., acyls), carboxylic acids (i.e., carboxyls), amides (i.e., amidos), imides (i.e., imidos), esters, anhydrides and the like.

The term “acryl” as used herein alone or in combination refers to a group represented by CH₂═C(O)C(O)O— where Q is an aliphatic or aromatic group.

The term “cyano,” “cyanate,” or “cyamide” as used herein alone or in combination refers to a carbon-nitrogren double bond. Representative examples of cyano groups include, but are not limited to, isocyanate, isothiocyanate and the like.

The term “amino” as used herein alone or in combination refers to a group containing a backbone nitrogen atom. Representative examples of amino groups include, but are not limited to, alkylamino, dialkylamino, arylamino, diarylamino, alkylarylamino, alkylcarbonylamino, arylcarbonylamino, carbamoyl, ureido and the like.

The term “phosphate-containing group” as used herein refers to a group containing at least one phosphorous atom in an oxidized state. Representative examples include, but are not limited to, phosphonic acids, phosphinic acids, phosphate esters, phosphinidenes, phosphinos, phosphinyls, phosphinylidenes, phosphos, phosphonos, phosphoranyls, phosphoranylidenes, phosphorosos and the like.

The term “sulfur-containing group” as used herein refers to a group containing a sulfur atom. Representative examples include, but are not limited to, sulfhydryls, sulfenos, sulfinos, sulfinyls, sulfos, sulfonyls, thios, thioxos and the like.

The term “optional” or “optionally” as used herein means that the subsequently described event or circumstance may or may not occur, and that the description includes instances where said event or circumstance occurs and instances where it does not. For example, the phrase “optionally substituted alkyl” means that the alkyl group may or may not be substituted and that the description includes both unsubstituted alkyl and alkyl where there is a substitution.

The term “effective amount,” when used in reference to a compound, product, or composition as provided herein, means a sufficient amount of the compound, product or composition to provide the desired result. The exact amount required will vary depending on the particular compound, product or composition used, its mode of administration and the like. Thus, it is not always possible to specify an exact “effective amount.” However, an appropriate effective amount may be determined by one of ordinary skill in the art informed by the instant disclosure using only routine experimentation.

The term “suitable” as used herein refers to a group that is compatible with the compounds, products, or compositions as provided herein for the stated purpose. Suitability for the stated purpose may be determined by one of ordinary skill in the art using only routine experimentation.

As used herein, the terms “administer” when used to describe the dosage of a compound, means a single dose or multiple doses of the compound.

As used herein, “apoptosis” refers to a form of cell death that includes progressive contraction of cell volume with the preservation of the integrity of cytoplasmic organelles; condensation of chromatin (i.e., nuclear condensation), as viewed by light or electron microscopy; and/or DNA cleavage into nucleosome-sized fragments, as determined by centrifuged sedimentation assays. Cell death occurs when the membrane integrity of the cell is lost (e.g., membrane blebbing) with engulfment of intact cell fragments (“apoptotic bodies”) by phagocytic cells.

As used herein, the term “cancer” means any condition characterized by resistance to apoptotic stimuli.

As used herein, the term “cancer treatment” means any treatment for cancer known in the art including, but not limited to, chemotherapy and radiation therapy.

As used herein, the term “combination with” when used to describe administration of an aminoacridine and an additional treatment means that the aminoacridine may be administered prior to, together with, or after the additional treatment, or a combination thereof.

As used herein, the term “treat” or “treating” when referring to protection of a mammal from a condition, means preventing, suppressing, repressing, or eliminating the condition. Preventing the condition involves treating the mammal prior to onset of the condition. Suppressing the condition involves treating the mammal after induction of the condition but before its clinical appearance. Repressing the condition involves treating the mammal after clinical appearance of the condition such that the condition is reduced or maintained. Elimination the condition involves treating the mammal after clinical appearance of the condition such that the mammal no longer suffers the condition.

As used herein, the term “tumor cell” means any cell characterized by resistance to apoptotic stimuli.

2. NF-kB-Mediated Mechanism of p53 Suppression in Tumors

The present invention is related to the discovery that p53 may be activated in those cancer cells that have functionally blocked p53 by inhibiting NF-κB activity. Inactivation of p53 pathway in tumors is a much broader phenomenon than p53 mutations. Even if a tumor maintains wild type p53, its function is usually either completely or partially lost. These cases are especially interesting from the therapeutic standpoint since p53 in such cancers can be viewed as a target for a pharmacological reactivation. There are some types of tumors in which p53 activity is blocked by tissue-specific mechanisms. Thus, Hdm2 overexpression is especially frequent in sarcomas, while E6 of human papilloma virus inactivates p53 in the majority of cervical carcinomas. RCC provides another example of that kind of tumor, which is especially interesting for the analysis since wild type p53 in RCC, as we recently reported, is repressed by an unknown dominant mechanism that is likely to be tissue specific. Hence, p53 reactivation seems to be an attractive strategy for treatment of this, so far, incurable form of cancer as well as other cancers with similar mechanisms for inactivating p53.

NF-κB activity is linked with the suppression of apoptosis in vitro and in vivo. Consistently, many apoptosis-resistant tumors acquire constitutive activation of NF-κB. Activation of NF-κB in tumor cells presumably contributes to their malignant phenotype by providing resistance to both natural (e.g., TNF, Fas or TRAIL) and pharmacological (chemotherapeutic drugs) death stimuli. While constitutively active NF-κB has been described in many tumor types, the connection between activation of NFκB and the inhibition of p53 has failed to be fully appreciated.

Cancers, such as those with functional or wild-type p53, may be treated by inhibiting NF-κB activity, which may lead to restoration of wild-type p53 activity and its activation. Inhibitors of NF-κB activity may also be used to sensitize cancers to p53-dependent and p53-independent apoptosis by treatments such as chemotherapeutics, radiotherapy or natural death ligands, such as TNF polypeptides. Regardless of their p53 status, the majority of human cancers have constitutively hyperactivated NF-κB. As a results, inhibitors of NF-κB may be used for treatment of any tumor regardless of their p53 status due to the reprogramming of transactivation NF-κB complexes into transrepression complexes.

3. Aminoacridines

Aminoacridines are representative examples of agents which may be used to inhibit NF-κB activity. The aminoacridine may be of the following formula: 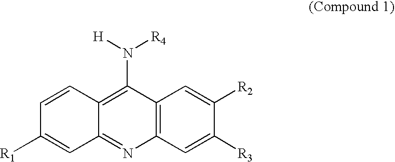
wherein,
R₁ is H or halogen;
R₂ is H or optionally substituted alkoxy;
R₃ is H or optionally substituted alkoxy; and
R₄ is H or optionally substituted aliphatic, aryl, or heterocycle.

Representative examples of aminoacridines include, but are not limited to, 9-aminoacridine or Mepacrine, which is otherwise known as Quinacrine, as well as those aminoacridines described in Example 2. The use of aminoacridines to sensitize tumor cells is attractive because many aminoacridines have limited side effects.

9AA has been used as therapeutic agent since 1942. Certain 9AA derivatives have been believed to be intercalating capable of DNA damaging activity; however, we found that 9AA and quinacrine did not show DNA damaging activity. Both 9aa and quinacrine were found to be more toxic to tumor than to normal cells in vitro and in vivo. Moreover, both compounds were shown to be capable of p53 activation and p53-dependent killing of a variety of tumor cell types, besides RCC. p53 dependence of their anti-tumor activity clearly distinguishes the aminoacridines from conventional chemotherapeutic drugs based on their targeting of tumors with wild type or functional p53.

Aminoacridines do not fit any known category of p53 activating agents. Although they may cause accumulation of p53, they do not induce p53 phosphorylation, unlike DNA damaging drugs. Moreover, aminoacridines do not cause DNA damage. Instead, the primary effect of aminoacridines appeared to be not p53 activation but repression of NF-κB, which later leads to p53 induction. Importantly, inhibition of NF-κB activates p53 function in a cell in which it cannot be “waked up” by any of the direct approaches to p53 activation, including introduction of Arf, knockdown of Hdm2 or ectopic overexpression of p53.

Inhibition of NF-κB is usually achieved through stabilization of the main negative regulator of NF-κB, IκB. Genetically, it can be done by mutating regulatory phosphorylation sites of this protein and pharmacologically—through inhibition of upstream kinases leading to a block of IκB phosphorylation. Many known chemical inhibitors of NF-κB act through this mechanisms. Stabilization of IκB results in cytoplasmic sequestration and functional inactivation of NF-κB complexes as transcription factors.

The activity of aminoacridines may be superior to previous drugs since they promotes strong accumulation of NF-κB complexes in the nuclei in response to activating stimuli accompanied with a complete repression of transactivation. Hence, aminoacridines may inhibit NF-κB by a mechanism acting downstream of IκB and involving conversion of NF-κB into an inactive complex. The lack of NF-κB-dependent transcription may lead to the depletion of the pool of IκB (that is a direct transcription target of NF-κB) and retention of NF-κB in the nucleus due to the lack of nuclear export, normally exerted by IκB. Interestingly, the knockout of any of the cellular factors involved in NF-κB activation (IKKα, IKKβ, TBK1, PKC-zeta) does not imitate the effect of aminoacridines, suggesting that none of them is a target of aminoacridines. It has recently demonstrated that nuclear accumulation of inactive NF-κB complexes, containing p65, occurs after cell treatment with UV, doxorubicin and daunorobicin; however, none of these treatments is comparable with aminoacridines in activating p53, presumably due to weaker NF-κB inhibitory activity.

The aminoacridines may be effective not only against the IκB phosphorylation arm of NF-κB signaling (“canonical” NF-κB activation pathway), but also through alternative mechanisms of NF-κB activation. This is supported by the ability of aminoacridines, such as 9AA, to block stimulated NF-κB activity and also effectively reduce basal levels of constitutive NF-κB activity in tumor cells. By contrast, IKK2 inhibitors are only able to block stimulated NF-κB activity.

4. Compositions

The present invention relates to a composition comprising an aminoacridine and optionally a chemotherapeutic. The present invention also relates to a composition comprising an aminoacridine and optionally a TNF polypeptide.

a. Chemotherapeutic

The chemotherapeutic may be any pharmacological agent or compound that induces apoptosis. The pharmacological agent or compound may be, for example, a small orgnanic molecule, peptide, polypeptide, nucleic acid, or antibody.

The chemotherapeutic may be a cytotoxic agent or cytostatic agent, or combination thereof. Cytotoxic agents prevent cancer cells from multiplying by: (1) interfering with the cell's ability to replicate DNA and (2) inducing cell death and/or apoptosis in the cancer cells. Cytostatic agents act via modulating, interfering or inhibiting the processes of cellular signal transduction which regulate cell proliferation and sometimes at low continuous levels.

Classes of compounds that may be used as cytotoxic agents include the following: alkylating agents (including, without limitation, nitrogen mustards, ethylenimine derivatives, alkyl sulfonates, nitrosoureas and triazenes): uracil mustard, chlormethine, cyclophosphamide (Cytoxan®), ifosfamide, melphalan, chlorambucil, pipobroman, triethylene-melamine, triethylenethiophosphoramine, busulfan, carmustine, lomustine, streptozocin, dacarbazine, and temozolomide; antimetabolites (including, without limitation, folic acid antagonists, pyrimidine analogs, purine analogs and adenosine deaminase inhibitors): methotrexate, 5-fluorouracil, floxuridine, cytarabine, 6-mercaptopurine, 6-thioguanine, fludarabine phosphate, pentostatine, and gemcitabine; natural products and their derivatives (for example, vinca alkaloids, antitumor antibiotics, enzymes, lymphokines and epipodophyllotoxins): vinblastine, vincristine, vindesine, bleomycin, dactinomycin, daunorubicin, doxorubicin, epirubicin, idarubicin, ara-c, paclitaxel (paclitaxel is commercially available as Taxol®), mithramycin, deoxyco-formycin, mitomycin-c, 1-asparaginase, interferons (preferably IFN-α), etoposide, and teniposide. Other proliferative cytotoxic agents are navelbene, CPT-11, anastrazole, letrazole, capecitabine, reloxafine, cyclophosphamide, ifosamide, and droloxafine.

Microtubule affecting agents interfere with cellular mitosis and are well known in the art for their cytotoxic activity. Microtubule affecting agents useful in the invention include, but are not limited to, allocolchicine (NSC 406042), halichondrin B (NSC 609395), colchicine (NSC 757), colchicine derivatives (e.g., NSC 33410), dolastatin 10 (NSC 376128), maytansine (NSC 153858), rhizoxin (NSC 332598), paclitaxel (Taxol®, NSC 125973), Taxol® derivatives (e.g., derivatives (e.g., NSC 608832), thiocolchicine NSC 361792), trityl cysteine (NSC 83265), vinblastine sulfate (NSC 49842), vincristine sulfate (NSC 67574), natural and synthetic epothilones including but not limited to epothilone A, epothilone B, and discodermolide (see Service, (1996) Science, 274:2009) estramustine, nocodazole, MAP4, and the like. Examples of such agents are also described in Bulinski (1997) J. Cell Sci. 110:3055 3064; Panda (1997) Proc. Natl. Acad. Sci. USA 94:10560-10564; Muhlradt (1997) Cancer Res. 57:3344-3346; Nicolaou (1997) Nature 387:268-272; Vasquez (1997) Mol. Biol. Cell. 8:973-985; and Panda (1996) J. Biol. Chem 271:29807-29812.

Also suitable are cytotoxic agents such as epidophyllotoxin; an antineoplastic enzyme; a topoisomerase inhibitor; procarbazine; mitoxantrone; platinum coordination complexes such as cis-platin and carboplatin; biological response modifiers; growth inhibitors; antihormonal therapeutic agents; leucovorin; tegafur; and haematopoietic growth factors.

Cytostatic agents that may be used include, but are not limited to, hormones and steroids (including synthetic analogs): 17.alpha.-ethinylestradiol, diethylstilbestrol, testosterone, prednisone, fluoxymesterone, dromostanolone propionate, testolactone, megestrolacetate, methylprednisolone, methyl-testosterone, prednisolone, triamcinolone, hlorotrianisene, hydroxyprogesterone, aminoglutethimide, estramustine, medroxyprogesteroneacetate, leuprolide, flutamide, toremifene, zoladex.

Other cytostatic agents are antiangiogenics such as matrix metalloproteinase inhibitors, and other VEGF inhibitors, such as anti-VEGF antibodies and small molecules such as ZD6474 and SU6668 are also included. Anti-Her2 antibodies from Genetech may also be utilized. A suitable EGFR inhibitor is EKB-569 (an irreversible inhibitor). Also included are Imclone antibody C225 immunospecific for the EGFR, and src inhibitors.

Also suitable for use as an cytostatic agent is Casodex® (bicalutamide, Astra Zeneca) which renders androgen-dependent carcinomas non-proliferative. Yet another example of a cytostatic agent is the antiestrogen Tamoxifen® which inhibits the proliferation or growth of estrogen dependent breast cancer. Inhibitors of the transduction of cellular proliferative signals are cytostatic agents. Representative examples include epidermal growth factor inhibitors, Her-2 inhibitors, MEK-1 kinase inhibitors, MAPK kinase inhibitors, PI3 inhibitors, Src kinase inhibitors, and PDGF inhibitors.

b. TNF Polypeptides

The TNF polypeptide may be a member of the TNF superfamily of ligands. Representative examples of TNF polypeptides include, but are not limited to, NGF, CD40L, CD137L/4-1BBL, TNF-α, CD134L/OX40L, CD27L/CD70, FasL/CD95, CD30L, TNF-β/LT-α, LT-β, and TRAIL. Members of the TNF superfamily are natural proteins that are implicated in the maintenance and function of the immune system and that can trigger apoptosis. The TNF polypeptide may be TRAIL, which induces apoptosis mainly in tumor but not in normal cells.

The activity of these so-called “death ligands” is believed to be mediated by binding with members of the TNF receptor family, which contain structurally similar death domains in their intracellular portions. Ligation of these receptors, specific for each death ligand, trigger activation of a cascade of events resulting in caspase activation. Representative examples of TNF-R receptors bound by the TNF polypeptides include, but are not limited to, LNGFR/p75, CD40, CD137/4-1BB/ILA, TNFRI/p55/CD120a, TNFRII/p75/CD120b, CD134/OX40/ACT35, CD27, Fas/CD95/APO-1, CD30/Ki-1, LT-β R, DR3, DR4, DR5, DcR1/TRID, TR2, GITR and osteoprotegerin.

Due to their unique ability to induce apoptosis in tumor cells, TNF family members are considered to be potential anticancer pharmaceuticals. However, many tumor cells escape pro-apoptotic action of death ligands, thereby reducing the use of these agents to death ligand-sensitive cancers and allowing the tumor to escape host immune response. The use of an inhibitor of NF-kB may be used to sensitize tumor cells to the killing of a death ligand, such as a TNF polypeptide.

It also contemplated that other agents may be used in the place of the TNF polypeptide. For example, an antibody may be used that mimics the activity of a TNF polypeptide. Representative examples of such antibodies include, but are not limited to, an agonist antibody to FAS, TRAIL receptor or TNFR. In addition, aptamers and other synthetic ligands capable to activate the corresponding receptors may be used.

c. Salts

The active agents of the compositions may be useful in various pharmaceutically acceptable salt forms. The term “pharmaceutically acceptable salt” refers to those salt forms which would be apparent to the pharmaceutical chemist, i.e., those which are substantially non-toxic and which provide the desired pharmacokinetic properties, palatability, absorption, distribution, metabolism or excretion. Other factors, more practical in nature, which are also important in the selection, are cost of the raw materials, ease of crystallization, yield, stability, hygroscopicity and flowability of the resulting bulk drug. Conveniently, pharmaceutical compositions may be prepared from the active ingredients or their pharmaceutically acceptable salts in combination with pharmaceutically acceptable carriers.

Pharmaceutically acceptable salts of the active agents include, but are not limited to, salts formed with a variety of organic and inorganic acids such as hydrogen chloride, hydroxymethane sulfonic acid, hydrogen bromide, methanesulfonic acid, sulfuric acid, acetic acid, trifluoroacetic acid, maleic acid, benzenesulfonic acid, toluenesulfonic acid, sulfamic acid, glycolic acid, stearic acid, lactic acid, malic acid, pamoic acid, sulfanilic acid, 2-acetoxybenzoic acid, fumaric acid, toluenesulfonic acid, methanesulfonic acid, ethanedisulfonic acid, oxalic acid, isethonic acid, and include various other pharmaceutically acceptable salts, such as, e.g., nitrates, phosphates, borates, tartrates, citrates, succinates, benzoates, ascorbates, salicylates, and the like. Cations such as quaternary ammonium ions are contemplated as pharmaceutically acceptable counterions for anionic moieties. In addition, pharmaceutically acceptable salts of the compounds of the present invention may be formed with alkali metals such as sodium, potassium and lithium; alkaline earth metals such as calcium and magnesium; organic bases such as dicyclohexylamine, tributylamine, and pyridine; and amino acids such as arginine, lysine and the like.

The pharmaceutically acceptable salts may be synthesized by conventional chemical methods. Generally, the salts are prepared by reacting the free base or acid with stoichiometric amounts or with an excess of the desired salt-forming inorganic or organic acid or base, in a suitable solvent or solvent combination.

In general, the counterions of the salts may be determined by the reactants used to synthesized the compounds. There may be a mixture of counterions of the salts, depending on the reactants. For example, where NaI is added to facilitate the reaction the counterion may be a mixture of Cl and I counter anions. Furthermore preparatory HPLC may cause the original counterion to be exchanged by acetate anions when acetic acid is present in the eluent. The counterions of the salts may be exchanged to a different counterion. The counterions are preferably exchanged for a pharmaceutically acceptable counterion to form the salts described above. Procedures for exchanging counterions are described in WO 2002/042265, WO 2002/042276 and S. D. Clas, “Quaternized Colestipol, an improved bile salt adsorbent: In Vitro studies.” Journal of Pharmaceutical Sciences, 80(2): 128-131 (1991), the contents of which are incorporated herein by reference. For clarity reasons, the counterions may not be explicitly shown in the chemical structures herein.

d. Formulations

The composition may further comprise one or more pharmaceutically acceptable additional ingredient(s) such as alum, stabilizers, antimicrobial agents, buffers, coloring agents, flavoring agents, adjuvants, and the like.

The composition may be in the form of tablets or lozenges formulated in a conventional manner. For example, tablets and capsules for oral administration may contain conventional excipients including, but not limited to, binding agents, fillers, lubricants, disintegrants and wetting agents. Binding agents include, but are not limited to, syrup, accacia, gelatin, sorbitol, tragacanth, mucilage of starch and polyvinylpyrrolidone. Fillers include, but are not limited to, lactose, sugar, microcrystalline cellulose, maizestarch, calcium phosphate, and sorbitol. Lubricants include, but are not limited to, magnesium stearate, stearic acid, talc, polyethylene glycol, and silica. Disintegrants include, but are not limited to, potato starch and sodium starch glycollate. Wetting agents include, but are not limited to, sodium lauryl sulfate). Tablets may be coated according to methods well known in the art.

The composition may also be liquid formulations including, but not limited to, aqueous or oily suspensions, solutions, emulsions, syrups, and elixirs. The composition may also be formulated as a dry product for constitution with water or other suitable vehicle before use. Such liquid preparations may contain additives including, but not limited to, suspending agents, emulsifying agents, nonaqueous vehicles and preservatives. Suspending agent include, but are not limited to, sorbitol syrup, methyl cellulose, glucose/sugar syrup, gelatin, hydroxyethylcellulose, carboxymethyl cellulose, aluminum stearate gel, and hydrogenated edible fats. Emulsifying agents include, but are not limited to, lecithin, sorbitan monooleate, and acacia. Nonaqueous vehicles include, but are not limited to, edible oils, almond oil, fractionated coconut oil, oily esters, propylene glycol, and ethyl alcohol. Preservatives include, but are not limited to, methyl or propyl p-hydroxybenzoate and sorbic acid.

The composition may also be formulated as suppositories, which may contain suppository bases including, but not limited to, cocoa butter or glycerides. The composition may also be formulated for inhalation, which may be in a form including, but not limited to, a solution, suspension, or emulsion that may be administered as a dry powder or in the form of an aerosol using a propellant, such as dichlorodifluoromethane or trichlorofluoromethane. The composition may also be formulated transdermal formulations comprising aqueous or nonaqueous vehicles including, but not limited to, creams, ointments, lotions, pastes, medicated plaster, patch, or membrane.

The composition may also be formulated for parenteral administration including, but not limited to, by injection or continuous infusion. Formulations for injection may be in the form of suspensions, solutions, or emulsions in oily or aqueous vehicles, and may contain formulation agents including, but not limited to, suspending, stabilizing, and dispersing agents. The composition may also be provided in a powder form for reconstitution with a suitable vehicle including, but not limited to, sterile, pyrogen-free water.

The composition may also be formulated as a depot preparation, which may be administered by implantation or by intramuscular injection. The composition may be formulated with suitable polymeric or hydrophobic materials (as an emulsion in an acceptable oil, for example), ion exchange resins, or as sparingly soluble derivatives (as a sparingly soluble salt, for example).

The composition may also be formulated as a liposome preparation. The liposome preparation can comprise liposomes which penetrate the cells of interest or the stratum corneum, and fuse with the cell membrane, resulting in delivery of the contents of the liposome into the cell. For example, liposomes may be used such as those described in U.S. Pat. No. 5,077,211, U.S. Pat. No. 4,621,023 or U.S. Pat. No. 4,508,703, which are incorporated herein by reference. A composition intended to target skin conditions can be administered before, during, or after exposure of the skin of the mammal to UV or agents causing oxidative damage. Other suitable formulations can employ niosomes. Niosomes are lipid vesicles similar to liposomes, with membranes consisting largely of non-ionic lipids, some forms of which are effective for transporting compounds across the stratum corneum.

5. Treatment

The composition may be used for treating a condition associated with NF-kB activity in vivo by administering to a patient in need thereof an aminoacridine. The NF-κB activity may be at any level, the reduction of which would lead to treatment of the condition. The NF-κB activity may also be at a basal level. The NF-κB activity may also be at a constitutive level. The NF-κB activity may also be at an induced constitutive level.

The condition associated with NF-kB activity may be cancer. A variety of cancers may be treated including, but not limited to, the following: carcinoma including that of the bladder (including accelerated and metastatic bladder cancer), breast, colon (including colorectal cancer), kidney, liver, lung (including small and non-small cell lung cancer and lung adenocarcinoma), ovary, prostate, testes, genitourinary tract, lymphatic system, rectum, larynx, pancreas (including exocrine pancreatic carcinoma), esophagus, stomach, gall bladder, cervix, thyroid, renal, and skin (including squamous cell carcinoma); hematopoietic tumors of lymphoid lineage including leukemia, acute lymphocytic leukemia, acute lymphoblastic leukemia, B-cell lymphoma, T-cell lymphoma, Hodgkins lymphoma, non-Hodgkins lymphoma, hairy cell lymphoma, histiocytic lymphoma, and Burketts lymphoma; hematopoietic tumors of myeloid lineage including acute and chronic myelogenous leukemias, myelodysplastic syndrome, myeloid leukemia, and promyelocytic leukemia; tumors of the central and peripheral nervous system including astrocytoma, neuroblastoma, glioma, and schwannomas; tumors of mesenchymal origin including fibrosarcoma, rhabdomyoscarcoma, and osteosarcoma; and other tumors including melanoma, xenoderma pigmentosum, keratoactanthoma, seminoma, thyroid follicular cancer, teratocarcinoma, renal cell carcinoma (RCC), pancreatic cancer, myeloma, myeloid and lymphoblastic leukemia, neuroblastoma, and glioblastoma.

Transformation induced by tax of HTLV, a causative agent of human adult T-lymphoblastic leukemia (ATL), may share the same molecular targets involved in RCC. For example, NF-kB is constitutively active in tax-transformed cells. Similar to RCC, p53 activity is inhibited through activation of NF-kB in tax-transformed cells and p53 inhibition does not involve sequestering of p300. Based on the shared mechanism of p53 inactivation, the compositions may also be used to treat HTLV-induced leukemia. Regardless of their p53 status, the majority of human cancers have constitutively hyperactivated NF-kB. The composition may also be capable of inhibiting NF-kB by reprogramming transactivation NF-kB complexes into transrepression complexes, which may also be used for treatment of any tumor regardless of their p53 status. The compositions may also be used for treating HIV infections since HIV LTRs are strongly dependent on NF-kB activity.

The composition may also be used as an adjuvant therapy to overcome anti-cancer drug resistance that may be caused by constitutive NF-kB activation. The anti-cancer drug may be a chemotherapeutic described herein.

a. Administration

The composition may be administered simultaneously or metronomically with other anti-cancer treatments such as chemotherapy and radiation therapy. The term “simultaneous” or “simultaneously” as used herein, means that the other anti-cancer treatment and the composition is administered within 48 hours, 24 hours, 12 hours, 6 hours, 3 hours or less, of each other. The term “metronomically” as used herein means the administration of the composition at times different from the chemotherapy and at certain frequency relative to repeat administration and/or the chemotherapy regiment.

The composition may be administered in any manner including, but not limited to, orally, parenterally, sublingually, transdermally, rectally, transmucosally, topically, via inhalation, via buccal administration, or combinations thereof. Parenteral administration includes, but is not limited to, intravenous, intraarterial, intraperitoneal, subcutaneous, intramuscular, intrathecal, and intraarticular. The composition may also be administered in the form of an implant, which allows slow release of the composition as well as a slow controlled i.v. infusion.

b. Dosage

A therapeutically effective amount of an agent required for use in therapy varies with the nature of the condition being treated, the length of time that activity is desired, and the age and the condition of the patient, and is ultimately determined by the attendant physician. The desired dose may be conveniently administered in a single dose, or as multiple doses administered at appropriate intervals, for example as one, two, three, four or more subdoses per day. Multiple doses often are desired, or required.

When given in combination with other therapeutics, the composition may be given at relatively lower dosages. In addition, the use of targeting agents may allow the necessary dosage to be relatively low. Certain compositions may be administered at relatively high dosages due to factors including, but not limited to, low toxicity, high clearance, low rates of cleavage of the tertiary amine. As a result, the dosage of a composition may be from about 1 ng/kg to about 200 mg/kg, about 1 μg/kg to about 100 mg/kg, or about 1 mg/kg to about 50 mg/kg. The dosage of a composition may be at any dosage including, but not limited to, about 1 μg/kg, 25 μg/kg, 50 μg/kg, 75 μg/kg, 100 μg/kg, 125 μg/kg, 150 μg/kg, 175 μg/kg, 200 μg/kg, 225 μg/kg, 250 μg/kg, 275 μg/kg, 300 μg/kg, 325 μg/kg, 350 μg/kg, 375 μg/kg, 400 μg/kg, 425 μg/kg, 450 μg/kg, 475 μg/kg, 500 μg/kg, 525 μg/kg, 550 μg/kg, 575 μg/kg, 600 μg/kg, 625 μg/kg, 650 μg/kg, 675 μg/kg, 700 μg/kg, 725 μg/kg, 750 μg/kg, 775 μg/kg, 800 μg/kg, 825 μg/kg, 850 μg/kg, 875 μg/kg, 900 μg/kg, 925 μg/kg, 950 μg/kg, 975 μg/kg, 1 mg/kg, 5 mg/kg, 10 mg/kg, 15 mg/kg, 20 mg/kg, 25 mg/kg, 30 mg/kg, 35 mg/kg, 40 mg/kg, 45 mg/kg, 50 mg/kg, 60 mg/kg, 70 mg/kg, 80 mg/kg, 90 mg/kg, or 100 mg/kg.

6. Diagnostic Methods

The composition may also be used to diagnose whether a tumor of a patient is capable of being treated by the composition. A sample of the tumor may be obtained from the patient. Cells of the tumor may then be transduced with a p53 reporter system, such as a p53-responstive lacZ reporter. The transduced cells may then be incubated with the composition. The production of a p53-mediated signal above controls indicates that the tumor may be treated by the composition.

7. Screening Methods

The present invention also relates to methods of identifying agents that modulate NF-κB activity. An agent that modulates NF-κB activity may be identified by a method comprising adding a candidate modulator of NF-κB activity to a cell-based NF-κB activated expression system, whereby a modulator of NF-κB activity is identified by the ability to alter the level of NF-κB activated expression. An agent that modulates NF-κB activity may also be identified by a method comprising adding a candidate modulator of NF-κB activity to a cell-based p53 activated expression system, whereby a modulator of NF-κB activity is identified by the ability to alter the level of p53 activated expression. An agent that modulates NF-κB activity may also be identified by a method comprising adding an aminoacridine and a candidate modulator of NF-κB activity to an NF-κB or p53 activated expression system, comparing the level of NF-κB or p53 activated expression to a control, whereby a modulator of NF-κB activity is identified by the ability to alter the level of NF-κB or p53 activated expression system compared to the control.

The cell may comprise a functionally silent p53. The cell may also comprise an NF-κB transactivation complex. The p53 activated expression system may be in a renal carcinoma cell line. The cell line may also be a sarcoma cell line. The cell line may also be a cell line with amplified mdm2. The cell line may also be a cell line that expresses HPV-E6 or is capable thereof.

Candidate agents may be present within a library (i.e., a collection of compounds). Such agents may, for example, be encoded by DNA molecules within an expression library. Candidate agent be present in conditioned media or in cell extracts. Other such agents include compounds known in the art as “small molecules,” which have molecular weights less than 10⁵ daltons, preferably less than 10⁴ daltons and still more preferably less than 10³ daltons. Such candidate agents may be provided as members of a combinatorial library, which includes synthetic agents (e.g., peptides) prepared according to multiple predetermined chemical reactions. Those having ordinary skill in the art will appreciate that a diverse assortment of such libraries may be prepared according to established procedures, and members of a library of candidate agents can be simultaneously or sequentially screened as described herein.

The screening methods may be performed in a variety of formats, including in vitro, cell-based and in vivo assays. Any cells may be used with cell-based assays. Preferably, cells for use with the present invention include mammalian cells, more preferably human and non-human primate cells. Cell-base screening may be performed using genetically modified tumor cells expressing surrogate markers for activation of NF-κB and/or p53. Such markers include, but are not limited to, bacterial β-galactosidase, luciferase and enhanced green fluorescent protein (EGFP). The amount of expression of the surrogate marker may be measured using techniques standard in the art including, but not limited to, colorimetery, luminometery and fluorimetery. Representative examples of cells that may be used in cell-based assays include, but are not limited to, renal cell carcinoma cells.

The conditions under which a suspected modulator is added to a cell, such as by mixing, are conditions in which the cell can undergo apoptosis or signaling if essentially no other regulatory compounds are present that would interfere with apoptosis or signaling. Effective conditions include, but are not limited to, appropriate medium, temperature, pH and oxygen conditions that permit cell growth. An appropriate medium is typically a solid or liquid medium comprising growth factors and assimilable carbon, nitrogen and phosphate sources, as well as appropriate salts, minerals, metals and other nutrients, such as vitamins, and includes an effective medium in which the cell can be cultured such that the cell can exhibit apoptosis or signaling. For example, for a mammalian cell, the media may comprise Dulbecco's modified Eagle's medium containing 10% fetal calf serum.

Cells may be cultured in a variety of containers including, but not limited to tissue culture flasks, test tubes, microtiter dishes, and petri plates. Culturing is carried out at a temperature, pH and carbon dioxide content appropriate for the cell. Such culturing conditions are also within the skill in the art.

Methods for adding a suspected modulator to the cell include, but are not limited to, electroporation, microinjection, cellular expression (i.e., using an expression system including naked nucleic acid molecules, recombinant virus, retrovirus expression vectors and adenovirus expression), use of ion pairing agents and use of detergents for cell permeabilization.

The present invention has multiple aspects, illustrated by the following non-limiting examples.

### EXAMPLES

### Materials and Methods

Cells

Renal cell carcinoma cell lines used, RCC45, RCC54 and ACHN are described in Gurova, et al. (2004). Cancer Res 64, 1951-1958. H1299, HT1080, MCF7, LNCaP, PC3, DU145, HCT116, SK-N-SH, WI38 cells were obtained from ATCC. The primary culture of normal kidney epithelial cells (NKE) was provided by J. Didonato (Cleveland Clinic Foundation, OH). 041 fibroblast cell line from Li-Fraumeni patient was provided by G. Stark. Mel7 and Mel29 cells are melanoma cell lines, described in Kichina, et al. (2003). Oncogene 22, 4911-4917. All cells were maintained in RPMI 1640 medium, supplemented with 10% FBS, 1 mM sodium pyruvate, 10 mM Hepes buffer, 55 nM β-mercaptoethanol and antibiotics.

Reporter cell lines with p53 responsive β-galactosidase were described in Gurova, et al. (2004). Cancer Res 64, 1951-1958. Reporter cell lines with p53 responsive luciferase was generated by transfection of p21-ConALuc plasmid with following selection on G418. Reporter cell lines with NF-κB-dependent luciferase were obtained by cotransfection of pNF-κBLuc and pEGFP-mito (Clontech) plasmids followed by selection on G418 (marker provided by pEGFP-mito plasmid). Reporter cell lines with myc, or Clock/Bmal responsive reporters were kindly provided by C. Burkhart and M. Antoch (Cleveland Clinic Foundation, OH).

Cells with inhibited p53 expression were generated by retroviral transduction of pBabeH1-sip53 or pBabeH1-siGFP vectors for siRNA expression followed by selection on puromycin.

Plasmids

p53, Arf expression vectors, pBabeH1-siHdm2, p21-ConALuc reporter plasmid are described in Gurova, et al. (2004). Cancer Res 64, 1951-1958. pNF-κBLuc plasmid was provided by N. Neznanov (Cleveland Clinic Foundation, ref. 59). pCDNA3 vector expressing pss-IκB was provided by I. Budunova (Northwestern University). pBabeH1-sip53 and pBabeH1-siGFP vectors for siRNA expression were generated by insertion of H1promoter and 64 oligonucleotide loop template for siRNA expression into left LTR of pBabeH1-puro vector analogously to pBabeH1-siHDM2 vector, described in Gurova, et al. (2004). Cancer Res 64, 1951-1958. Sequences for siRNA against p53 and GFP are described in Brummelkamp, et al. (2002). Science 296, 550-553. Lentiviral plasmids for p53 or GFP expression are described in Gurova, et al. (2004). Cancer Res 64, 1951-1958.

Chemicals

DiverSet library of 34,000 chemical compounds was obtained from Chembridge, Inc. Focused libraries of around 30d9 and 9AA were provided by Chembridge, Inc. All other chemicals were obtained from Sigma.

Retroviral and Lentiviral Transduction

Packaging cells (A293 from Clontech) plated in 60 mm plates were transfected with 2 μg of retroviral vector DNA using Lipofectamin Plus (Invitrogen) according to manufacturers recommendations. The medium was changed after 8 hours. Virus-containing media supplied with 8 μg/ml of Polybrene (Sigma) was collected at 24 and 48 hours post-transfection and used for infection. Virus-transduced cells were selected for the resistance to an appropriate selective agent (G418, hygromycin or puromycin, depending on the vector) up to a complete death of non-infected cells.

Stocks of recombinant lentiviruses carrying p53 or EGFP (control vector) were prepared using 293 cell line transfected with pLV-CMV-p53 and pLV-CMV-EGFP plasmids along with packaging plasmids encoding viral structural proteins and G-protein of vesicular stomatitis virus using lipofectamine reagent (Invitrogen). Virus-containing media from 293T cells was collected 48 hours later and transferred to target cells in the presence of 4 μg/ml of polybrene and virus was concentrated 50-100 times by ultracentrifugation. Virus titers (typically 10⁸ IU/ml) were determined by infection of Rat1a cells (that are known to be resistant to ectopic expression of p53), followed by selection on puromycin and counting colonies.

Chemical Library Screening

2×10⁴ of RCC45ConALacZ cells were plated into wells of 96 well plates in 200 μL of phenol-red free RPMI medium with standard additives. After overnight incubation library of chemical compounds in DMSO solution together with controls was added with the help of plastic bacterial replicators (200+/−100 nL). Final concentration of compounds was around 5 μg/ml. Negative control was DMSO, positive control was doxorubicin solution (0.2, 0.6 and 2 μM). After 24 hours lysis buffer with ONPG was added directly to the medium on ice. After 3 hours of incubation at 37° C., β-galactosidase activity was estimated by reading absorbance values Wallack 1420 plate reader (Perkin Elmer) at λ=430 nm. All compounds, inducing ONPG reaction stronger than the most effective concentration of doxorubicin was considered as primary hits.

Reporter Assays

For cotransfection set-up, 2×10⁵ cells were plated into 6 well plates and, after overnight incubation, transfected with Lipofectamin Plus reagent (Gibco BRL) with 0.5 μg of reporter plasmids (p21-ConALuc or pNF-κBLuc) in combination with different concentrations of p53, Arf, Ss-IκB, or siHDM2 expressing plasmids. Corresponding empty vectors were added into all transfections up to 2 μg of total DNA amount. Normalization of transfection efficiency was done by adding 0.2 μg of pCMV-LacZ plasmid. Luciferase activity and β-galactosidase activity was measured in lysates prepared 48 hours after transfection with Cell Lysis Buffer (Promega) by luciferase assay system (Promega) or β-galactosidase enzyme system (Promega). Luminometric and colorimetric reactions were read on the Wallack 1420 plate reader (Perkin Elmer). Integrated reporter set-up. 2×10⁴ of cells with integrated reporter were plated in 96 well plates. After overnights incubation chemical compounds or media from lentivirus producing cells were added. At different time points cell lysates were prepared using Reporter Lysis Buffer (Promega). Luciferase or β-galactosidase activity and protein concentration were measured in aliquots of cell lysates using standard kits (Promega, Luciferase and β-galactosidase assay systems, Biorad Protein Assay Kit).

Cell Survival Assays

5×10³ of cells were plated in 6 well plates and treated with different concentrations of drugs for 24 hours. Then fresh drug-free medium was added. Number of colonies was estimated after 5-6 days of incubation. Cell survival was estimated as a percentage of intensity of methylene blue staining of treated cells, comparing with untreated control (methylene blue from stained colonies was extracted by 0.1% of SDS and quantitated spectrophotometrically).

Cell Cycle Analysis

10⁶ of cells were plated in 100 mm plates and after overnight incubation, different concentrations of 9AA or doxorubicin were added. At the end of incubation period, cells were collected, fixed and stained with propidium iodide. DNA content was measured using FACScalibur (Becton Dickinson) and analyzed using CellQuest software.

Western Blot Analysis

Cells were lysed in RIPA buffer (25 mM Tris HCl, pH7.2, 125 mM NaCl, 1% NP40, 1% sodium deoxycholate, 1 mM EDTA) containing 1 mM PMSF (Sigma), 10 μg/ml of aprotinin (Sigma) and 10 μg/ml of leupeptin (Sigma). Protein concentrations were determined with BioRad Dc protein assay kit. Equal protein amounts were run on gradient 4-20% precast gels (Novex) and blotted onto PVDF membranes (Amersham). The following antibodies were used: anti-p53—monoclonal mouse DO1 (Santa-Cruz), anti-p21—monoclonal mouse F-5 (Santa-Cruz), anti-mdm2—monoclonal mouse SMP14 (Santa-Cruz). p53 phosphorylation status was analyzed using phospho-p53 sampler kit from Cell signaling according to manufacturer's recommendations, anti-p65—(C20, Santa Cruz), anti-phospho-p65—(ser536, Cell Signaling), anti-IκBa—(C21, Santa Cruz), anti-p50—(NLS, Santa Cruz). HRP-conjugated secondary antibodies were purchased from Santa-Cruz. Quantitation of the data was performed using Quantity One (BioRad).

Immunofluorescent Immunostaining

Cells in chamber slides were washed with PBS and fixed consequently with 10% phosphate buffered formalin at room temperature, 100% methanol at −20° C. and acetone at −20° C. Then slides were blocked in the solution of 3% BSA, 0.1% Triton X100 in PBS for 1 hour. Anti-p65 antibodies (C-20, Santa-Cruz) were added in concentration of 1 μg/ml in blocking solution. Secondary anti-rabbit Cy2 conjugated antibodies (Sigma) was used. All washings were done with blocking solution.

DNA—Topoisomerase II Activity Assay

HT1080 cells were labeled for 24 hours with 0.02 to 0.04 mCi/mL of [¹⁴C] thymidine, specific activity 53 mCi/mmol (Amersham). The labeled HT1080 cells were treated with different concentrations of etoposide (VP-16), amsacrine (m-AMSA), or 9-aminoacridine for 1 h. The induction of topo II-mediated DNA scission was determined by measuring precipitation of the protein DNA complex using a modification of the SDS-KCl technique.

Proteasome Inhibitor Assay

The proteasome assay kit was purchased from Boston Biochem, Inc. and used according to the manufacturer's recommendations.

Electromobility Shift Assay (EMSA)

Nuclear extracts were prepared as described in Chernov, et al. (1997). Oncogene 14, 2503-2510. Annealed oligonucleotide, corresponding to NF-κB binding site (Santa-Cruz), was radio-labeled by [α³²P]dCTP by Klenow polymerase and then by [γ-³²P]dATP by T4 polynucleotide kinase. 10⁷ cpm of labeled oligonucleotide was affinity purified on Probe Quant columns (Amersham). Radio-labeled oligonucleotide was added to 10 μg of protein nuclear extract together with 1 μg of poly-dIdC (Amersham) to prevent nonspecific binding and incubated for 30 minutes at room temperature. For supershift, 200 ng of anti-p65, anti-p50 or anti-antibodies were added to the reaction (all antibodies are from Santa Cruz). After 30 minutes incubation, the entire reaction mixtures were loaded into 4% polyacrilamide gel in 0.5×TBE buffer and run for 2 hours at 200V. Dried gels were exposed to X-ray films for 30 minutes-1 hour.

Animal Experiments

NIH Swiss athymic nude, male mice, 5-6 weeks old were purchased from Harlan. 5×10⁶ of tumor cells were inoculated into the flank of mice in 100 μL of PBS. When tumors reached 5 mm diameter, intraperitoneal injections of drugs were started in 100 μl solution of 50% DMSO in PBS (except quinacrine, which was dissolved in PBS). As vehicle, 50% DMSO solution in PBS was used. Tumor size was measured in three dimensions every other day.

### Example 1

### RCC Cell-Based Readout for Isolation of P53-Activating Agents

The transactivation function of p53 is inhibited in RCC cells by an unknown inhibitory factor, suggesting drug-mediated restoration of p53 function as an approach to selective killing of this type of tumor cells as well as other tumor cells with similar inhibition of p53. To test whether the reactivation of p53 would be toxic for RCC cells, we ectopically expressed p53 in five RCC-derived cell lines in an attempt to deplete the inhibitory factor. Cells were supplemented with integrated p53-responsive reporter (ConALacZ) to monitor p53 reactivation. p53 cDNA was transduced using a lentiviral vector with CMV promoter. p53-deficient lung adenocarcinoma cell line H1299, which are sensitive to wild type p53, and rat fibroblastoid cell line Rat1, which is resistant to human p53, were used as controls.

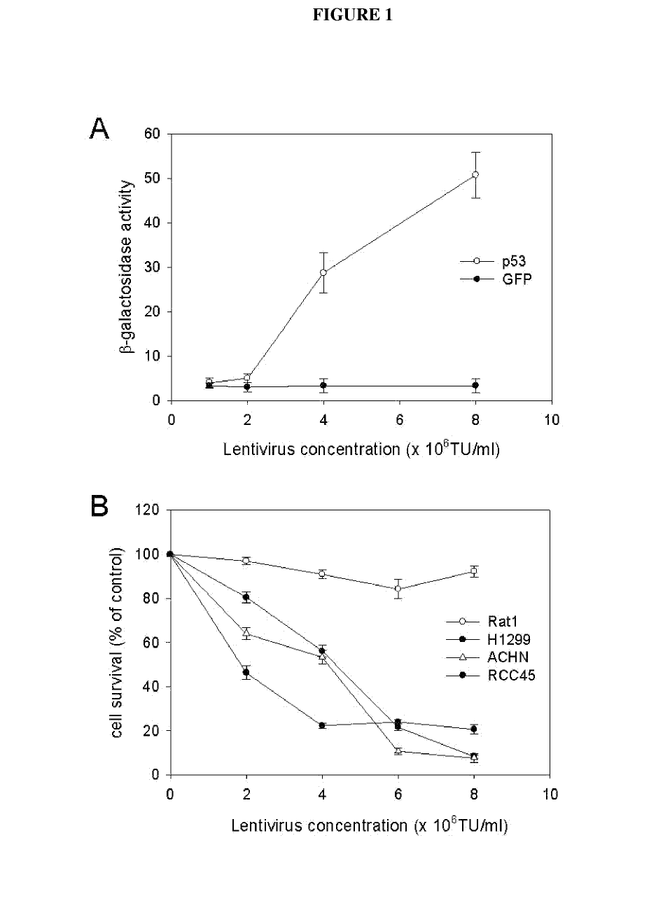

As shown in FIG. 1, dormant p53 in RCC may be reactivated, and that reactivation leads to tumor cell death. Starting from a certain level of expression, p53 became simultaneously cytotoxic and active in inducing the reporter in RCC45 cells (FIGS. 1a and b). This indicates that. cells, such as RCC cells, may be used in a cell-based reporter system to screen for agents that are capable of reactivating p53. This also indicates that reactivation of p53 in tumor cells, such as RCC cells, may be cytotoxic.

### Example 2

### Screening Chemical Library Identity Aminoacridines as a Potent P53 Activator in RCC

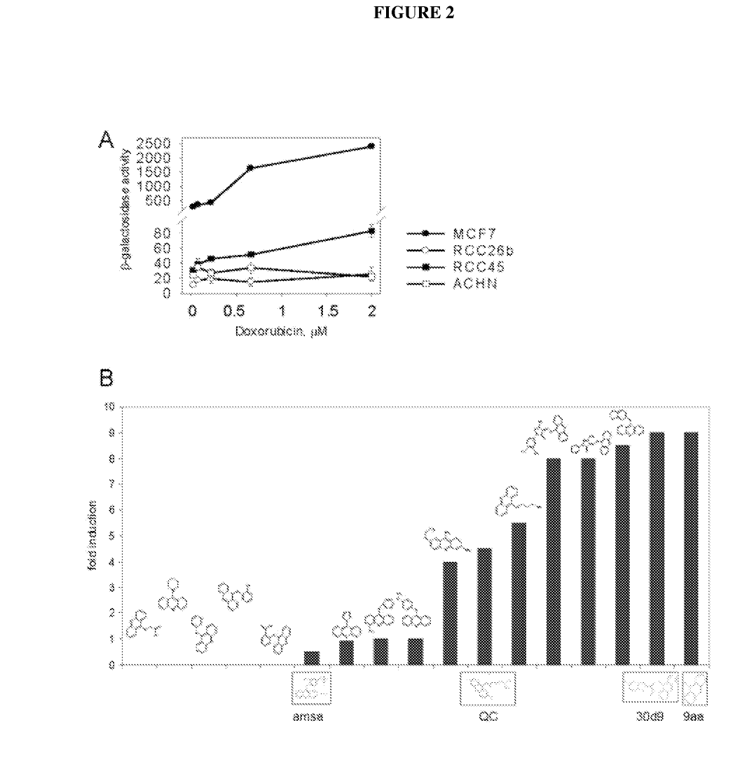

We carried out a direct cell-based screening of chemicals capable of restoring p53 transactivation in RCC with hope to isolate small molecules with therapeutic potential that could also be used as tools for deciphering mechanisms of RCC specific p53 repression. RCC45ConALacZ cells were used to screen a diverse chemical library of 34,000 compounds (Chembridge Corporation). β-galactosidase activity was measured in cell lysates 24 hours after incubation with the compounds. Twenty-eight compounds that induced β-galactosidase activity higher than that of 1 μM of doxorubicin were considered as primary hits (FIG. 2a). The most active compound (compound 30d9) caused 22-fold induction of the reporter in RCC45 cells acting 7 times stronger than doxorubicin. The library of structural analogues built around compound 30d9 and consisting of 40 chemicals was screened using the same cell-based reporter assay. Two agents of the formula of compound 1 were found to be active.

A library of 59 derivatives of compound 30d9 were then screened, including the anti-cancer agent amsacrine (amsa) and anti-malaria agent quinacrine. Twelve of the tested compounds reactivated p53 in RCC45 cells ranging in their activity similar to doxorubicin (e.g., amsa) to 7-10 folds stronger than doxorubicin, with compound 30d9 and 9AA being the strongest (FIG. 2b). Quinacrine showed an intermediate level of activity. SAR analysis indicated that aminoacridines are capable of reactivating p53.

### Example 3

### Aminoacridines are a Potent Activator of P53 in a Variety of Cell Types

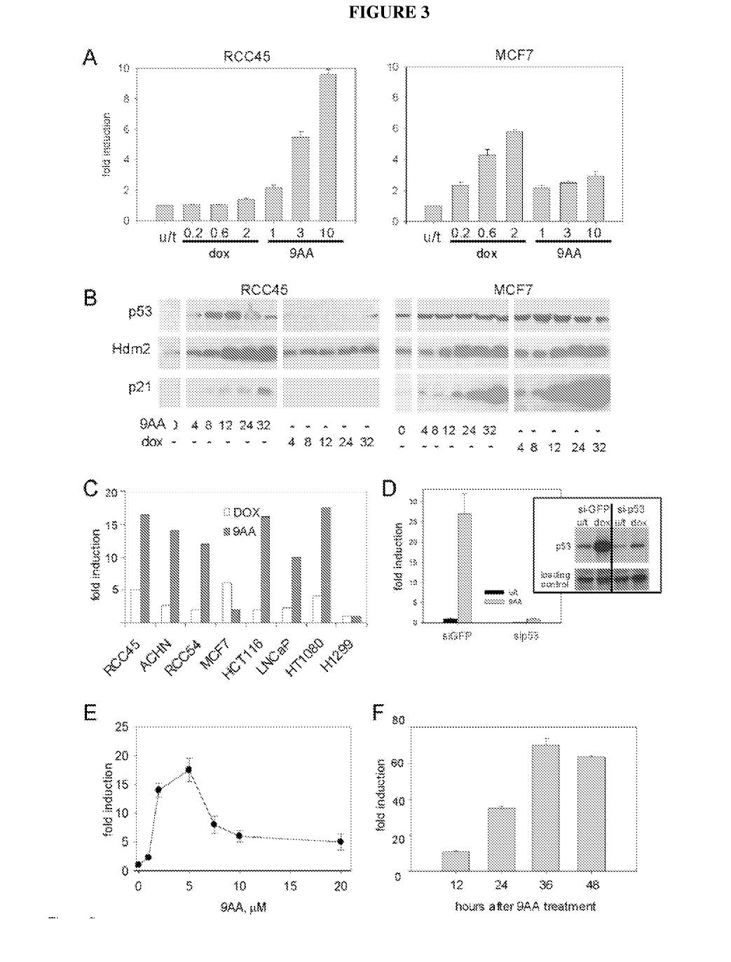

9AA was much stronger than doxorubicin not only in the induction of the p53 reporter gene in RCC45 cells, but also of endogenous p53-responsive genes as judged by the analysis of protein levels of p21/Waf1 and Hdm2 (FIGS. 3a and b). Interestingly, that in MCF7 cells, in which the p53 pathway is very active 9AA effects were weaker than that of doxorubicin (FIGS. 3a and b).

We tested the effect of 9AA on p53 transactivation in several other reporter cell lines with wild type p53. In the majority of cells tested, 9AA stimulated reporter activity much stronger than doxorubicin or other DNA-damaging drug used (FIG. 3c and data not shown), suggesting that a common mechanism may be involved in the negative control of active p53 in different tumor cell types. The strongest induction of p53 by 9AA was observed in human fibrosarcoma HT1080 cells, which we used in many assays in parallel with RCC cell lines. The effect of 9AA was p53-dependent since no stimulation of reporter activity was detected in p53 null H1299 cells or in cells with p53 expression inhibited by siRNAs (FIGS. 3c and d).

9AA activated p53 at concentrations as low as 1 μM (FIG. 3e). The kinetics of activation is unusually slow as compared with DNA-damaging stimuli: 9AA-induced p53-dependent transactivation becomes detectable only 12 hours and reaches maximum around 36 hours after treatment with the drug (FIG. 3f). One-hour incubation with 9AA was enough to initiate p53 activation being detectable several hours later (FIG. 3f).

### Example 4

### Aminoacridines are Toxic for Tumor Cells with Wild Type P53

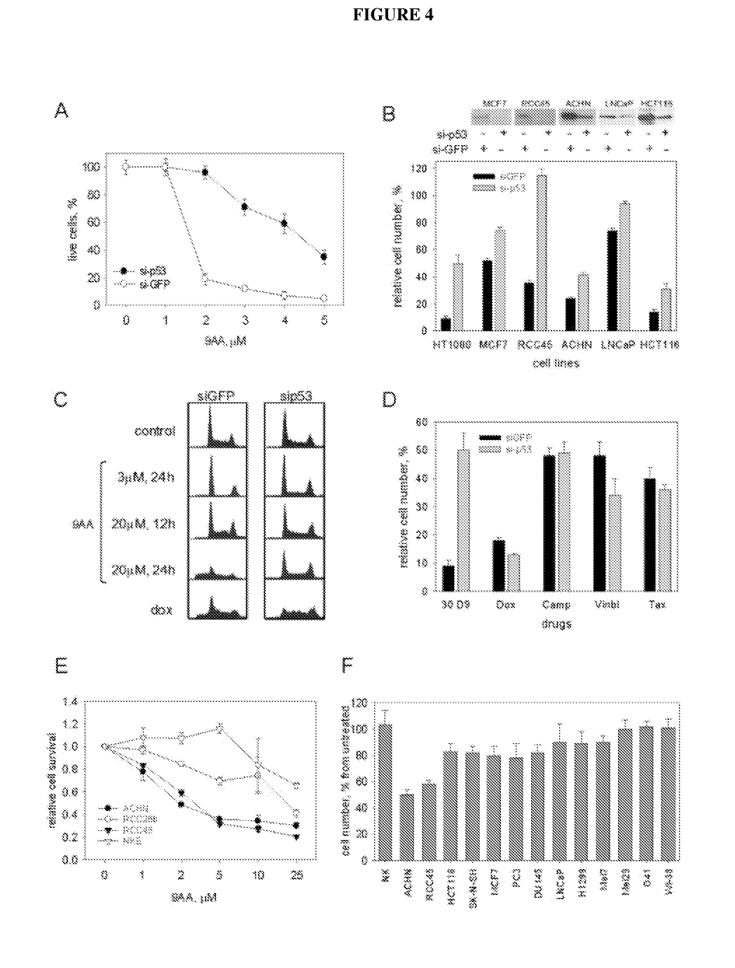

To test whether activation of p53 is translated into p53-dependent cytotoxicity, we compared the effect of 9AA on the survival of cells differing in p53 status. For this purpose we generated a series of isogenic pairs of cell lines expressing either anti-p53 or control siRNA-expressing constructs. The degree of p53 downregulation by siRNA was tested by western immunoblotting. For all cell variants tested, 9AA was found to be toxic. However, it was less toxic to the cells with reduced p53 expression with maximum difference observed in HT1080 cells (FIG. 4 a & b).

Depending on the dose, 9AA caused p53-dependent growth arrest (3 μM) or apoptosis (20 μM, FIG. 4c). Low doses of 9AA did not cause apoptosis even after longer incubation (up to 48 hours), while high doses of the compound induced apoptosis with no prior growth arrest. Only marginal changes in cell cycle distribution were observed in 9AA-treated p53-deficient cells, as opposed to doxorubicin which caused more pronounced alterations in distribution of cells among phases of the cell cycle in p53 deficient than in control cells presumably due to lack of G1 checkpoint control (FIG. 4c).

Interestingly, the toxicity of none of the chemotherapeutic drugs acting through a DNA-damaging mechanism (e.g., camptothecin, doxorubicin) or by affecting the microtubule network (e.g., taxol, vinblastine) was found to be p53-dependent (FIG. 4d), suggesting that aminoacridines kill tumor cells through a mechanism different from conventional chemotherapeutic agents. Since normal cells also possess active p53, we tested the toxicity of 9AA to normal kidney epithelial cells and human diploid fibroblasts WI38. Both normal cell types were more resistant to 9AA as compared with RCC and other tumor cells (FIGS. 4e and f).

### Example 5

### Aminoacridine-Based Drugs Have Anti-Tumor Effects In Vivo

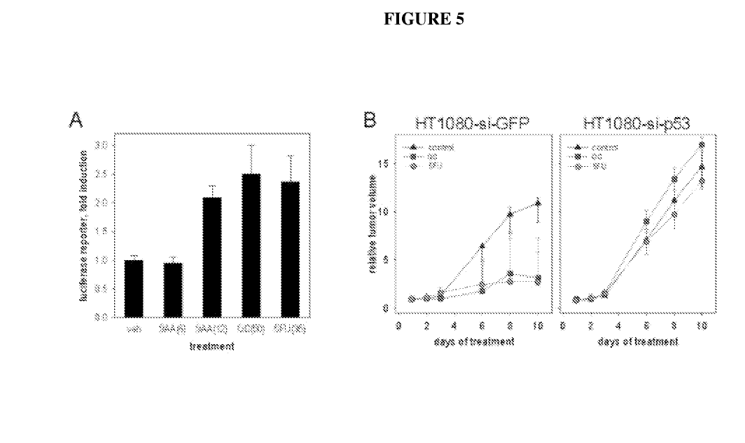

The in vivo anti-tumor effects of aminoacridines were tested in a xenograft tumor model using HT1080 cells differing in their p53 status and carrying p53 luciferase reporters grown subcutaneously in nude mice. The activity of the p53-dependent luciferase reporter in tumor cells was used for testing the bioavailability of 9AA in vivo. Both 9AA and quinacrine were capable of activating p53 in tumors (FIG. 5a) and both compounds showed similar properties in the above-described experiments (data not shown).

Mice were inoculated with HT1080sip53 (left flank) and HT1080siGFP (right flank) cells to exploit p53 dependence of the quinacrine effect. After tumors reached 5 mm in diameter, mice were injected i.p. daily with 50 mg/kg of quinacrine, with 5-fluorouracil (5FU, 35 mg/kg) being used for comparison. As presented in FIG. 5b, quinacrine inhibited growth of p53-expressing tumors to the same extend as 5FU and had no effect on the growth of p53-deficient tumors. This indicates that aminoacridines inhibit the in vivo growth of xenograft tumors in a p53 dependent manner (FIG. 5b).

Mice were also inoculated with 2×10⁶ human prostate cancer cells using PC3 p53-negative or with DU145 cells comprising mutant p53. The mice were treated with quinacrine at a dose of 100 mg/kg by oral gavage or with sterile water, as a control. Tumor growth was reduced by ˜50% in quinacrine-treated mice. Interestingly, this indicates that aminoacridines also inhibit in vivo growth of xenograft tumors in a p53-independent manner.

### Example 6

### Mechanism of Aminoacridine-Mediated Activation of P53

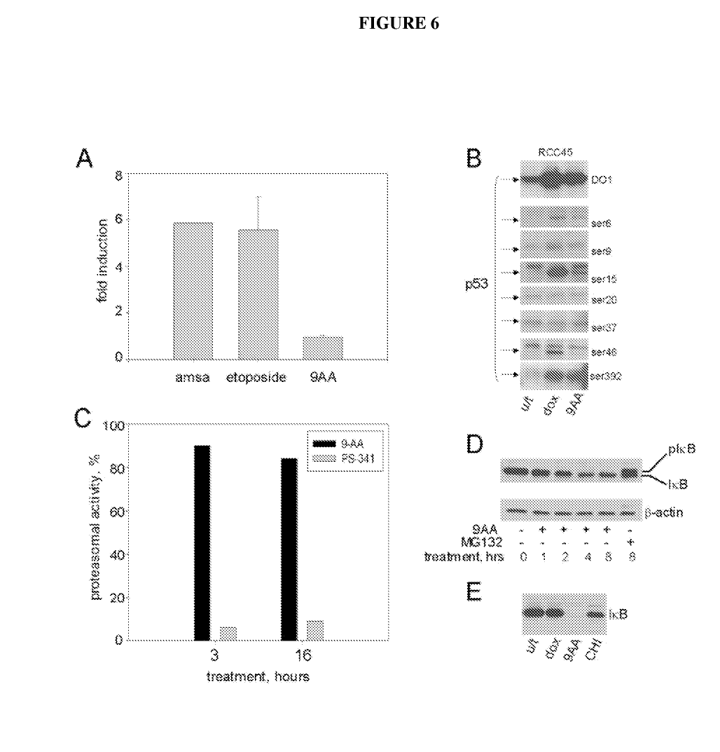

We first tested whether aminoacridines activate p53 through DNA damage, a well-known mechanisms of action of p53 inducing compounds. Since one of the 9AA derivatives, amsa, causes DNA damage via poisoning of topoisomerase II, we tested and found no effect of 9AA on topoisomerase II activity in vitro, in contrast to amsa and etoposide, another inhibitor of topoisomerase II (FIG. 6a). Moreover, 9AA did not affect p53 phosphorylation status, as may be expected from DNA damaging drugs that activate p53 through DNA break-sensitive kinases (ATM, ATR etc). Doxorubicin, used in these experiments as a positive control, did induce phosphorylation of p53, confirming that upstream p53 signaling is functional in RCC (FIG. 6b). The results, together with previous results showing that different types of DNA damage did not activate p53 in RCC, indicate that aminoacridines activate p53 through a mechanism different from DNA damage.

Proteasome inhibitors form another class of p53-activating agents causing accumulation of unmodified p53 protein. Accumulation of non-phosphorylated p53 in response to 9AA treatment and predominant localization of the drug in the cytoplasm (monitored by fluorescent microscopy) suggested that aminoacridines could act as an inhibitor of proteasomal degradation. This hypothesis was ruled out using a direct in vitro assay (FIG. 6c) and by monitoring the effect of 9AA on the level of IκB, another target of proteasomal degradation [21], which was used as an independent indicator of proteasomal activity. While MG132 (inhibitor of proteasomal degradation used as positive control) caused accumulation of phosphorylated forms of IkB, 9AA treatment surprisingly had the opposite effect leading to a gradual decrease followed by complete disappearance of IκB (FIGS. 6d and e). These results indicate that aminoacridines do not activate p53 by proteasome inhibition.

### Example 7

### Aminoacridines Inhibit NF-κB

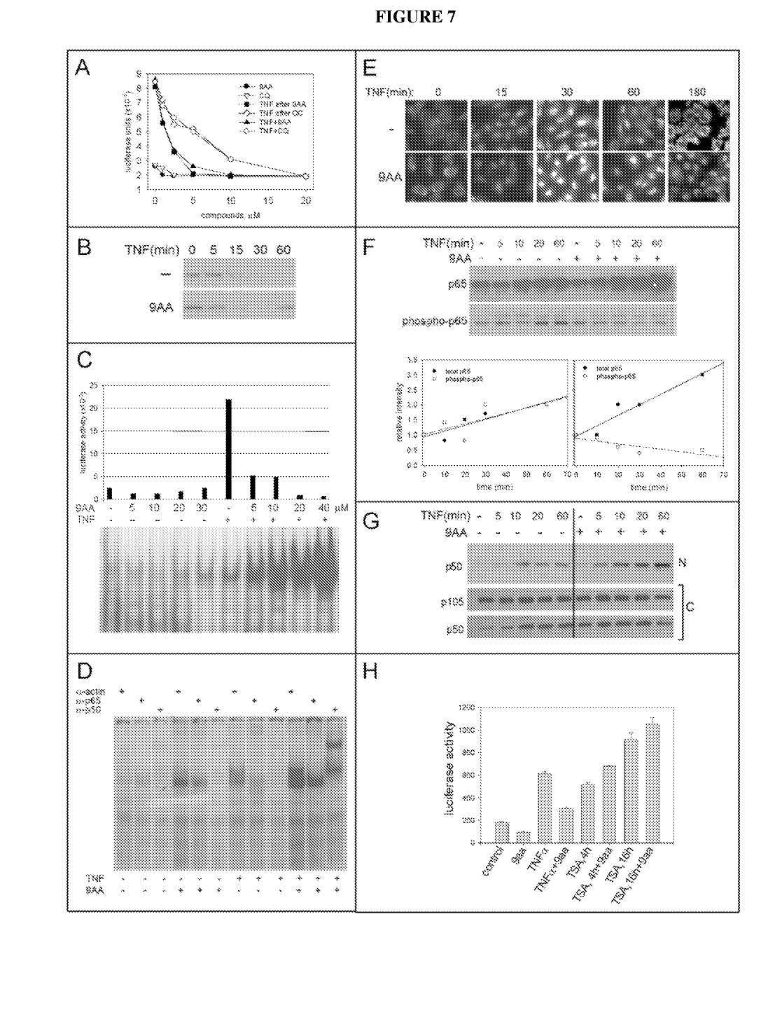

As shown in Example 6, 9AA unexpectedly induced degradation of IκB. Based on these results, we focused on the possible effects of aminoacridines on the NF-κB pathway. We used p53-null H1299 cells with an integrated NF-κB-responsive reporter to test the effect of 9AA on the transcriptional activity of NF-κB. We found that 9AA and quinacrine both inhibited basal and TNF-induced activity of the reporter (FIG. 7a). They were effective being added before (−24-Oh), simultaneously or after (0-6 hours) TNF stimulation (data not shown). They also inhibited TNF-stimulated induction of IκB, an essential part of the NF-κB feedback regulatory loop (FIG. 7b). Surprisingly, blocking NF-κB-dependent transactivation by 9AA and quinacrine coincided with a simultaneous dose dependent increase in DNA-binding activity of NF-κB (FIG. 7c). Some increase was observed even without TNF stimulation which predominantly involved p50/p50 homodimer (FIG. 7d). This increase was especially strong when the drugs were applied in combination with TNF, involving both p65/p50 and p50/p50 NF-κB complexes (FIGS. 7c and d).

The increase in DNA binding activity of NF-κB complexes correlated with nuclear accumulation of p65-containing NF-κB complexes upon TNF stimulation, in the presence of 9AA, as observed by immunofluorescent staining. We found that p65 enters the nuclei regardless of the presence of 9AA, but the drug significantly prolongs the time of its presence in the nuclei (FIG. 7e), presumably due to the lack of induction of IκB that serves as a shuttle exporting NF-κB from the nucleus.

These results indicate that aminoacridines are a potent inhibitor of NF-κB transactivation that acts through an unusual mechanism. As opposed to previously described inhibitors that act by stabilizing IκB, aminoacridines may act by converting NF-κB complex into a transcriptionally inactive state that becomes trapped in the nucleus due to lack of induction of its shuttling factor IκB.

Among the mechanisms that could be responsible for functional inactivation of NF-κB complex could be a lack of phosphorylation of p65 (reported as essential for the NF-κB activity) resulting in recruitment of histone deacetylases (HDAC) into the complex and conversion of chromatin in the transcription initiation sites into inactive form. To test this possibility, we analyzed the effect of 9AA on p65 phosphorylation in the nuclei of cells treated with TNF. In the cells treated with TNF alone, the proportion of phosphorylated p65 increased in parallel with total p65. In the cells treated with TNF and 9AA, the proportion of phosphorylated p65 did not increase (FIG. 7f). These results show that in the presence of aminoacridines, p65 enters the nuclei predominantly in an under-phosphorylated state and, therefore, presumably inactive form.

Inactive NF-κB is present in the nuclei of unstimulated cells in a complex with HDACs. The inhibition of HDAC activity by trichostatin A (TSA) results in the activation of NF-κB-dependent transcription without any additional stimuli. If inactivation of NF-κB by aminoacridines also involves HDACs, it should have no effect on TSA-stimulated induction of NF-κB. In fact, treatment of NF-κB reporter cells with TSA resulted in significant activation of NF-κB-dependent transcription that could not be blocked by 9AA (FIG. 7h). These results show that aminoacridines may cause accumulation of inactive NF-κB complexes and their inactivation involves HDAC.

Blocking phosphorylation of p65 may not be the only mechanism of anti-NF-κB activity of aminoacridines. We found that treatment with 9AA caused an increase in cytoplasmic and nuclear pools of p50 (FIG. 7g). p50/p50 homodimers, the proportion of which increased with 9AA-mediated accumulation of this NF-κB subunit (FIG. 7d), might also contribute to the formation of inactive NF-κB complexes.

### Example 8

### Aminoacridines Activate P53 in RCC Through Inhibition of NF-κB

Aminoacridines cause two strong effects in RCC cells. They activate p53 in an unusual way that does not involve DNA damage or inhibition of proteasomal degradation. It also inhibits NF-κB, also in an unusual way, by converting transcriptionally active NF-κB complexes into transcriptionally inactive ones, presumably due to the inhibition of p65/RelA phosphorylation. The effects of aminoacridines on p53 and NF-κB are quite specific since they have no effect on transcription regulated by other tested factors, such as c-Myc or N-Myc, androgen receptor or CLOCK/BMAL1 (data not shown). We were interested to know if p53 activation and NF-κB repression by aminoacridines are related or distinct activities of the drugs, and, if interrelated, which one is the primary event. We could readily exclude the possibility of p53 activation being the driver of NF-κB repression since all the effects of aminoacridines on the NF-κB pathway were seen in p53-deficient (H1299) as well as in p53 wild type (HT1080) cells. Moreover, inhibition of NF-κB by aminoacridines is detectable within one hour after application of the compound, while their effects on p53 are unusually slow and require 12-16 hours of treatment to start seeing the effect (FIG. 2d). To explore the opposite model (NF-κB repression by aminoacridines drives p53 activation) we: (i) tested what happens with p53 activity when NF-κB is inhibited by an alternative mechanism and (ii) analyzed the effect of aminoacridines on p53 activation in NF-κB-deficient cells.

To further confirm the duel effect of 9AA and quinacrine on p53 and NF-kB, we analyzed changes in global gene expression profiles of two RCC cell lines, RCC45 and RCC54, treated with two does of 9AA (2 μM and 10 μM, causing growth arrest and apoptosis, respectively), using microarray hybridization. RNA was isolated after 16 hours of treatment that was enough to induce p53 but before appearance of signs of cell death. Among the 36847 genes represented on the array, only 0.6% changed their mRNA abundance by at least a factor of 2 at least in one dose in both cell lines. The most upregulated genes included p21, mdm2, as well as several other p53 targets (Table 1), while IκB alpha, IL-8 and several chemo- and cytokines (Table 2), all encoded by NF-kB-responsive genes, were strongly suppressed as a result of treatment. These results confirmed a dual effect of aminoacridines as an inducer of p53 and an inhibitor of NF-kB transcription.

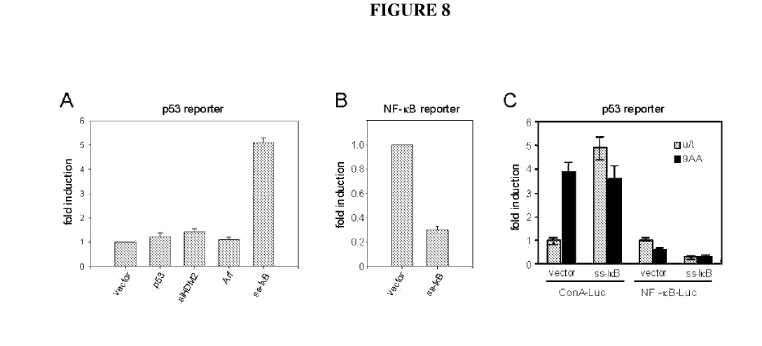

To suppress NF-κB activity in RCC cells, we used a stable IκB mutant lacking both phosphorylation sites, IκB super suppressor (ss-IκB). Transduction of this mutant into RCC ACHN cells resulted in a 3-fold inhibition of NF-κB reporter activity (FIG. 8a) that is consistent with our observations that NF-κB is constitutively active in all RCC cell lines tested. Importantly, the activity of the p53-responsive reporter in the same cells was increased up to 5 times upon transduction of NF-κB inhibitory ss-IκB (FIG. 8b). Similar effect was observed in another RCC line, RCC54, as well as in non-RCC cells HT1080 (data not shown), both responding to 9AA by p53 activation (FIG. 3a). Remarkably, ss-IκB activated p53 in RCC much stronger than transduction of “direct” regulators of p53 pathway, such as Arf, siRNA to Hdm2 or p53 itself (FIG. 8b). Thus, we have demonstrated that blocking NF-κB activity can restore p53 transactivation in RCC cells and that aminoacridines are likely to act through this mechanism.

To test whether NF-κB inhibition by aminoacridines is solely responsible for its p53 activating effect, we treated the RCC cell with NF-κB inhibited by ss-IκB by 9AA. We were unable to generate RCC cell lines stably expressing ss-IκB since inhibition of NF-κB interfered with RCC cell viability (unpublished observation). Therefore, the effect of aminoacridines on the cells with suppressed NF-κB was tested in transient transfection experiments, in which introduction of ss-IκB was combined with either NF-κB or p53 responsive reporters. Transfection of ss-IκB into ACHN inhibited NF-κB reporter activity down to the levels when treatment with 9AA did not cause any additional inhibition. Under these conditions, 9AA was incapable of activating p53 responsive reporter any further (FIG. 8c), indicating that p53-activating effect of aminoacridines is mediated via inhibition of NF-κB function and that NF-κB activity is responsible for p53 repression in RCC.

### Example 9

### Sensitization of Resistant Tumor Cells

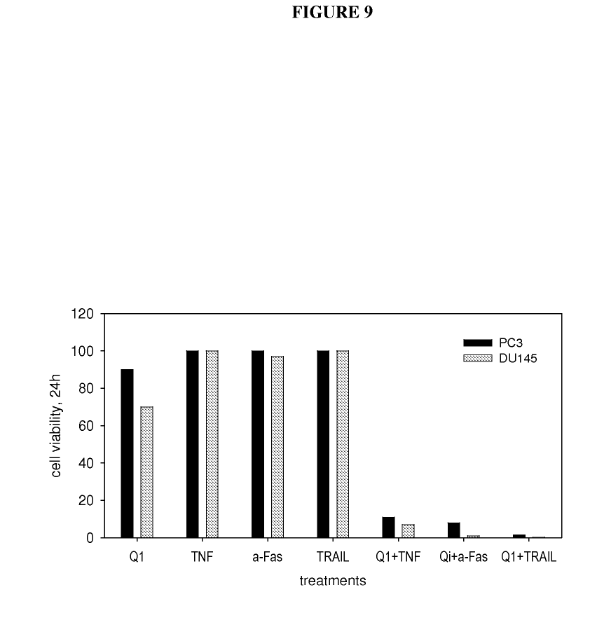

We next tested the ability of aminoacridines to sensitize tumor cells that are resistant to death ligands. We found that tumor cells of prostate, renal, lung and breast origin that originally were resistant to treatment with death ligand, were dramatically sensitized to TNF, Fas and TRAIL if treated in the presence of non-toxic or low toxic concentrations of aminoacridines (FIG. 9).

### Example 10

### Anti-Tumor Applications of Aminoacridines

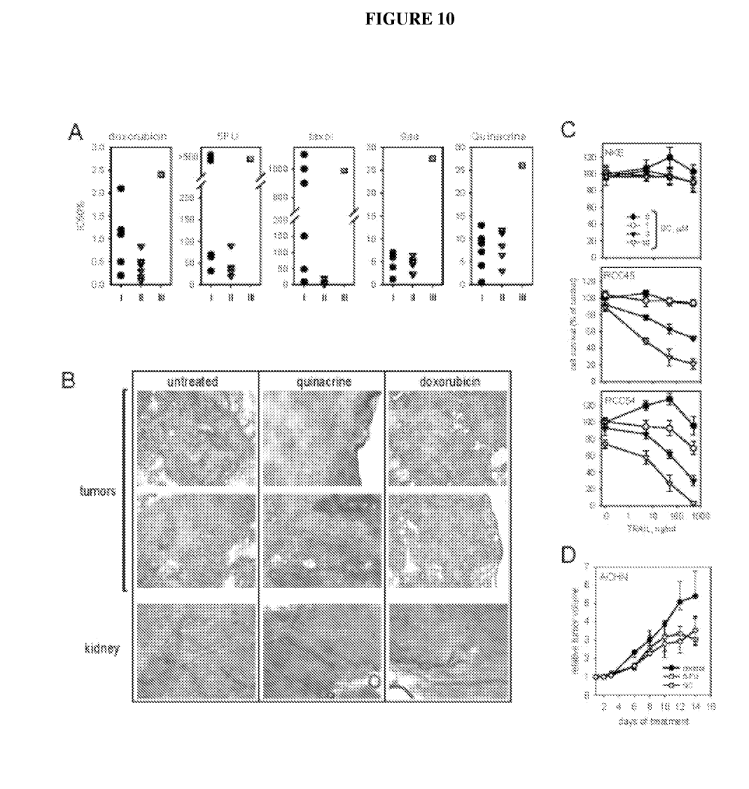

We tested whether the potency of the aminoacridines to conventional chemotherapeutics. For this purpose, we compared IC50% of several chemotherapeutics (doxorubicin, taxol and 5-fluorouracil), 9AA and quinacrine (QC) for a set of RCC and non-RCC derived tumor cells (6 of each type) as well as normal kidney cells (NKE). The non-RCC cell lines included: MCF7 (wt p53), HT1080 (wt p53); H1299 (p53 null), U20S (wt p53); LNCaP (wt p53), and HCT116 (wt p53). Average IC50% of RCC was higher than that of non-RCC cell lines to all chemotherapeutic agents used, and close to the IC50% of normal cells, which is in line with clinical reports (FIG. 10). 9AA and quinacrine effectively restored p53-mediated transactivation in 9 out of 10 RCC cell lines tested (data not shown). The compounds were equally efficient against both RCC and non-RCC cells regardless of p53 status of the latter. In addition, see Table 3.

We next verified that the aminoacridines would cause a similar effect in ex vivo organ cultures. Fresh RCC specimens and fragments of normal kidney from the same patients were placed in short-term organ cultures in the media containing lentiviral vector carrying p53-responsive lacZ reporter (FIG. 10B) for 24 hours. The tissue samples were then treated with quinacrine or doxorubicin for an additional 24 hours, then fixed with glutaraldehyde and stained for β-galactosidase activity using X-gal (blue staining). As shown in FIG. 10B, quinacrine but not doxorubicin treatment induced expression of the p53-responsive reporter in tumor samples; no reporter induction by either drug was detectable in normal kidney samples (normal kidney structures expressing endogenous β-galactosidase activity are seen); no X-gal positive staining was observed in any of the tumor samples that were not transduced with the reporter. Hence, p53 function was restored by aminoacridines. This assay cold also be used as the basis for a prognostic test that may predict tumor responsiveness to treatment aminoacridines.

Combination of two important properties in one compound, inhibition of NF-kB activity and activation of p53, offers potential application of aminoacridines. Resistance to tumor cells to TRAIL one of the most promising natural anticancer cytokines, is usually associated with constitutively active NF-kB and inhibition of p53, which controls expression of one of the TRAIL receptors, DR5. 9AA was capable of induction of TRAIL receptor (DR5) expression in two RCC cell lines tested (Table 1). Thus, compounds capable of simultaneous inhibition of NF-kB and activation of p53 are expected to reverse tumor cell resistance to TRAIL. We tested this by treating TRAIL resistant RCC cell lines RCC54 and RCC45 in combination with quinacrine. Normal kidney epithelial cells were used for comparison. As shown in FIG. 10C, quinacrine inverted TRAIL resistance of tumor but not normal kidney cells, indicating another mode of anticancer application of aminoacridines.

Finally, we tested the effect of quinacrine on the growth of tumor xenografts formed by ACHN cells s.c. injected in nude mice and found about 50% inhibition of tumor growth similar to the effect observed with 5-fluorouracil (5FU), but without significant weight loss (up to 20%) that accompanied 5FU application (FIG. 10D).

TABLE 1

| | Relative expression | | | | |
|---|---|---|---|---|---|
| | compared to untreated cells | | | | |
| | RCC54 | | RCC45 | | |
| gene name | 2 μM | 10 μM | 2 μM | 10 μM | references |
| Cyclin-dependent kinase inhibitor 1A (p21, | 1.25 | 3.80 | 1.33 | 4.62 | Rev. by Nakamura. 2004. |
| Cip1) | | | | | Cancer Sci. 95: 7 |
| Mdm2, transformed 3T3 cell double minute | 0.96 | 2.86 | 0.91 | 2.30 | |
| 2, p53 binding protein (mouse) | | | | | |
| Growth arrest and DNA-damage-inducible, | 1.22 | 2.78 | 0.92 | 2.66 | |
| beta | | | | | |
| Ribonucleotide reductase M2 B (TP53 | 1.11 | 3.53 | 1.08 | 3.33 | Ceballos E, et al. Oncogene. |
| inducible) | | | | | 2005 Apr 18; |
| Annexin A1 | 1.01 | 3.04 | 1.13 | 2.02 | Kannan et al. 2001. |
| | | | | | Oncogene 20: 3449 |
| Leucine-rich repeats and death domain | 1.12 | 2.12 | 1.10 | 1.64 | Lin et al. 2000. Nat Genet. |
| containing | | | | | 26: 122 |
| Tumor protein p53 inducible nuclear | 1.03 | 1.61 | 1.09 | 1.86 | Okamura et al. 2001. Mol. |
| protein 1 | | | | | Cell. 8: 85 |
| Apoptotic protease activating factor | 0.78 | 1.27 | 0.85 | 1.72 | Fortin et al. 2001. J. Cell. |
| | | | | | Biol. 155: 207 |
| Heat shock 27 kDa protein 1 | 0.82 | 1.30 | 1.13 | 1.71 | Gao et al. 2000. Int. J. |
| | | | | | Cancer. 88: 191 |
| KILLER/DR5 | 1.1 | 1.9 | 1.1 | 2.4 | Takimoto and El-Deiry 2000. |
| | | | | | Oncogene 19: 1735 |
| BCL2 binding component 3 (PUMA) | 1.03 | 1.57 | 0.78 | 1.79 | Nakano and Vousden. 2001. |
| | | | | | Mol. Cell. 7: 683 |
| Damage-specific DNA binding protein 2, | 0.89 | 1.38 | 1.00 | 1.79 | Kannan et al. Oncogene. |
| 48 kDa | | | | | 2001. 20: 2225 |
| Carboxylesterase 2 (intestine, liver) | 0.80 | 1.75 | 1.04 | 3.17 | |
| Thiosulfate sulfurtransferase (rhodanese) | 1.18 | 3.59 | 1.13 | 2.79 | |
| Amyloid beta (A4) precursor-like protein 2 | 1.19 | 1.68 | 0.97 | 1.90 | |
| Activating transcription factor 3 | 1.07 | 3.56 | 0.91 | 2.99 | |
| BTG family member 2 | 1.44 | 1.88 | 0.9 | 2.4 | |

TABLE 2

| | Relative expression | | | | |
|---|---|---|---|---|---|
| | compared to untreated cells | | | | |
| | RCC54 | | RCC45 | | |
| gene name | 2 μM | 10 μM | 2 μM | 10 μM | references |
| Chemokine (C-X-C motif) ligand 1 | 0.40 | 0.08 | 0.93 | 0.15 | Loukinova et al. Int J |
| (melanoma growth stimulating activity, alpha) | | | | | Cancer. 2001. 94: 637 |
| Interleukin 8 | 0.62 | 0.09 | 1.37 | 0.30 | Kunsch et al. J Immunol. |
| | | | | | 1994. 153: 153 |
| Chemokine (C-X-C motif) ligand 6 | 1.22 | 0.11 | 0.94 | 0.22 | Loukinova et al. Int J |
| (granulocyte chemotactic protein 2) | | | | | Cancer. 2001. 94: 637 |
| Chemokine (C-C motif) ligand 20 | 0.64 | 0.26 | 1.06 | 0.35 | Carson et al. Cancer Res. |
| | | | | | 2004. 64: 2096 |
| Tenascin C (hexabrachion) | 0.70 | 0.59 | 1.37 | 0.58 | Mettouchi et al. Mol Cell |
| | | | | | Biol. 1997. 17: 3202 |
| Nuclear factor of kappa light polypeptide | 0.73 | 0.54 | 0.96 | 0.55 | Hinz et al. J Exp Med. |
| gene enhancer in B-cells inhibitor, alpha | | | | | 2002. 196: 605 |
| Cyclin D1 (PRAD1: parathyroid | 0.77 | 0.29 | 1.34 | 0.69 | Guttridge et al. Mol Cell |
| adenomatosis 1) | | | | | Biol. 1999. 19: 5785 |
| V-myc myelocytomatosis viral oncogene | 1.19 | 0.40 | 0.88 | 0.48 | Bourgarel-Rey et al. Mol |
| homolog (avian) | | | | | Pharmacol. 2001. 59: 1165 |
| Chemokine-like factor super family 3 | 0.87 | 0.56 | 0.85 | 0.59 | Mettouchi et al. Mol Cell |
| | | | | | Biol. 1997. 17: 3202 |
| Tumor necrosis factor, alpha-induced | 1.26 | 0.48 | 0.73 | 0.27 | |
| protein 6 | | | | | |
| Tumor necrosis factor receptor superfamily, | 0.63 | 0.85 | 0.65 | 0.91 | Kim et al. FEBS Lett. 2003. |
| member 9 | | | | | 541: 163 |
| Vascular cell adhesion molecule 1 | 0.88 | 0.53 | 1.18 | 0.51 | Iademarco et al. J Biol |
| | | | | | Chem. 1992. 267: 16323 |
| Chemokine (C-C motif) ligand 2 | 0.64 | 0.26 | 1.06 | 0.35 | Martin et al. Eur J |
| | | | | | Immunol. 1997. 27: 1091 |
| CD44 antigen (homing function and Indian | 0.88 | 0.65 | 1.32 | 0.41 | Hinz et al. J Exp Med. |
| blood group system) | | | | | 2002. 196: 605 |

TABLE 3
Aminoacridines: combination with anti-cancer drugs

| drugs | ACHN | RCC54 | RCC45 | MCF7siGFP | MCF7sip53 | HT1080siGFP | HT1080sip53 | H1299 |
|---|---|---|---|---|---|---|---|---|
| 9aa | 1.7^{a} | 1.8 | 2.3 | 1.1 | 1.6 | 1.2 | 2.4 | |
| QC | 1.8 | 2.0 | 2.4 | 1.2 | 1.8 | 1.3 | 2.4 | |
| 5FU | 74 | NT | NT | 150 | 175 | 37 | 70 | |
| 9aa^{b} − 24 h | 77 | NS | NS | 110 | 183 | 28 | 77 | |
| 9aa^{c} 0 h | 94 | NS | NS | 130 | 167 | 30 | 79 | |
| 9aa^{d} + 24 h | 90 | NS | NS | 132 | 188 | 35 | 76 | |
| QC − 24 h | 79 | NS | NS | 107 | 176 | 34 | 77 | |
| QC 0 H | 86 | NS | NS | 113 | 177 | 34 | 73 | |
| QC + 24 h | 88 | NS | NS | 109 | 170 | 37 | 77 | |
| etoposide | 1.02 | NT | 1.8 | 0.3 | 1.05 | 2.1 | 1.1 | |
| 9aa − 24 h | 1.04 | NS | 1.9 | 0.6 | 0.95 | 2.0 | 1.1 | |
| 9aa 0 h | 1.00 | NS | 3 | 0.55 | 1.05 | 2.0 | 1.3 | |
| 9aa + 24 h | 1.06 | NS | 1.9 | 0.34 | 1.0 | 2.1 | 1.2 | |
| QC − 24 h | 1.1 | NS | 1.8 | 0.3 | 1.03 | 2.05 | 1.1 | |
| QC 0 H | 1.05 | NS | 2.8 | 0.27 | 1.1 | 1.9 | 1.1 | |
| QC + 24 h | 1.04 | NS | 1.7 | 0.29 | 1.09 | 1.95 | 1.2 | |
| paclitaxel | 0.08 | 60 | 55 | 0.05 | 6.0 | 0.08 | 0.08 | |
| 9aa − 24 h | 0.07 | 58 | 54 | 0.04 | 5.9 | 0.08 | 0.07 | |
| 9aa 0 h | 0.07 | 60 | 63 | 0.05 | 6.0 | 0.08 | 0.08 | |
| 9aa + 24 h | 0.07 | 59 | 61 | 0.05 | 6.05 | 0.08 | 0.08 | |
| QC − 24 h | 0.08 | 60 | 55 | 0.045 | 6.0 | 0.08 | 0.08 | |
| QC 0 H | 0.08 | 59 | 65 | 0.05 | 6.0 | 0.08 | 0.09 | |
| QC + 24 h | 0.08 | 56 | 60 | 0.055 | 6.0 | 0.08 | 0.08 | |

^{a}LD50%: concentration of a drug (μM) causing death of 50% of cells;
^{b}1 μM of 9aa or quinacrine (QC) was added 24 hours before a drug;
^{c}1 μM of 9aa or quinacrine (QC) was added 24 hours simultaneously with a drug;
^{d}1 μM of 9aa or quinacrine (QC) was added 24 hours after a drug.
NT—non-toxic in the concentration range used.
NS—no sensitization to non-toxic drugs by either of aminoacridines.

1. A method of treating a condition associated with NF-κB activity comprising administering to a patient in need thereof a composition comprising an inhibitor of NF-κB.

2. The method of claim 1, wherein the NF-κB activity is constitutive or induced.

3. The method of claim 1, wherein the NF-κB activity is at a basal level.

4. The method of claim 1, wherein inhibition of NF-κB activates p53.

5. The method of claim 1, wherein the condition is cancer.

6. The method of claim 5, wherein the inhibition of NF-κB leads to activation of functionally impaired wild type p53.

7. The method of claim 5, wherein the cancer is selected from the group consisting of renal cell carcinoma, sarcoma, prostate cancer, breast cancer, pancreatic cancer, myeloma, myeloid and lymphoblastic leukemia, neuroblastoma, glioblastoma and a cancer caused by HTLV infection.

8. The method of claim 1, wherein the condition is inflammation, an autoimmune disease, graft versus host disease, or a condition associated with HIV infection.

9. The method of claim 1, wherein the condition is pre-cancerous cells which have acquired dependence on constitutively active NF-κB.

10. The method of claim 1, wherein the inhibitor of NF-κB is an aminoacridine of the formula: 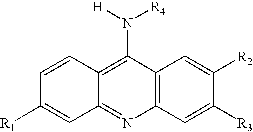
wherein,
R₁ is H or halogen;
R₂ is H or optionally substituted alkoxy;
R₃ is H or optionally substituted alkoxy; and
R₄ is H or optionally substituted aliphatic, aryl, or heterocycle.

11. The method of claim 10, wherein the aminoacridine is selected from the group consisting of 9-aminoacridine and quinacrine.

12. The method of claim 10, wherein the composition further comprises an activator of a death receptor of a TNF family polypeptide.

13. The method of claim 12, wherein the activator is a TNF family polypeptide selected from the group consisting of NGF, CD40L, CD137L/4-1BBL, TNF-α, CD134L/OX40L, CD27L/CD70, FasL/CD95, CD30L, TNF-β/LT-α, LT-β, and TRAIL.

14. A method of screening for an agent that activates functionally silent p53 comprising:
(a) adding a candidate agent to a cell comprising a p53-responsive reporter;
(b) measuring the level of signal of the p53-responsive reporter, whereby an agent is identified by signal in (b) above a control.

15. The method of claim 14 wherein the cell comprises a functionally silent p53.

16. A method of screening for an agent that inhibits NF-κB comprising:
(a) adding a candidate agent to a cell comprising a p53-responsive reporter;
(b) measuring the level of signal of the p53-responsive reporter, whereby an agent is identified by signal in (b) above a control.

17. The method of claim 16 wherein the cell comprises a functionally silent p53.

18. The method of claim 16 wherein the cell comprises an NF-κB transactivation complex.
